(19) United States
(12) Patent Application Publication
(10) Pub. No.: US 2022/0356520 A1
(43) Pub. Date: Nov. 10, 2022

(54) METHOD OF DIGITAL MULTIPLEX DETECTION AND/OR QUANTIFICATION OF BIOMOLECULES AND USE THEREOF

(71) Applicants: CENTRE NATIONAL DE LA RECHERCHE SCIENTIFIQUE (CNRS), Paris (FR); INSTITUT NATIONAL DE LA SANTE ET DE LA RECHERCHE MEDICALE (INSERM), Paris (FR); ECOLE SUPERIEURE DE PHYSIQUE ET DE CHIMIE INDUSTRIELLES DE LA VILLE DE PARIS, Paris (FR); PARIS SCIENCES ET LETTRES, Paris (FR); UNIVERSITE DE PARIS, Paris (FR)

(72) Inventors: Guillaume GINES, PARIS (FR); Yannick RONDELEZ, PARIS (FR); Thomas JET, PARIS (FR); Valérie TALY, BOURG LA REINE (FR)

(73) Assignees: CENTRE NATIONAL DE LA RECHERCHE SCIENTIFIQUE (CNRS), Paris (FR); INSTITUT NATIONAL DE LA SANTE ET DE LA RECHERCHE MEDICALE (INSERM), Paris (FR); ECOLE SUPERIEURE DE PHYSIQUE ET DE CHIMIE INDUSTRIELLES DE LA VILLE DE PARIS, Paris (FR); PARIS SCIENCES ET LETTRES, Paris (FR); UNIVERSITE DE PARIS, Paris (FR)

(21) Appl. No.: 17/614,023

(22) PCT Filed: May 27, 2020
(86) PCT No.: PCT/EP2020/064773

§ 371 Date: Nov. 24, 2021

(30) Foreign Application Priority Data
May 27, 2019 (EP) 19305670.2

(51) Int. Cl. C12Q 1/6876

(52) U.S. Cl. CPC C12Q 1/6876; C12Q 2600/16; C12Q 2600/178; C12Q 2600/112; C12Q 1/6834

(57) ABSTRACT

The present invention relates to a digital multiplex method for detecting and/or quantifying multiple target biomolecules in a sample, said biomolecules being selected from DNA, RNA, and proteins. The present invention further relates to different applications of the digital multiplex method and to a kit.

### FIELD OF THE INVENTION

The present invention relates to a digital multiplex method for detecting and/or quantifying multiple target biomolecules such as DNA, RNA and proteins in a sample, based on isothermal amplification having specific molecular design. The present invention further relates to a method for diagnosis of diseases selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases and to a method for agro diagnosis. It also relates to methods for detections of biomarkers (biomolecules) in food agri-food field and in the environment. All of these diagnosis and detection methods comprise the use of the digital multiplex method of the invention. The present invention also relates to a kit for detecting and/or quantifying multiple target biomolecules, said kit comprising particles functionalized with oligonucleotides, enzymes and separating agents.

### BACKGROUND OF THE INVENTION

Several biomolecules such DNA, RNA or proteins are used as attractive diagnostic, prognostic or predictive biomarkers. These molecules, and particularly the nucleic acids have accumulated clinical evidences that they are closely related to various diseases (cancers, neuronal or cardiovascular diseases, diabetes, etc.). Moreover, they are present in the body fluids and thus accessible via minimally invasive liquid biopsies (serum, plasma, urine). Circulating biomarkers can be assessed repeatedly enabling a regular follow up of treatments and relapses, large scale population screening or early diagnostic.

However, the use of circulating nucleic acids, particularly RNA and more particularly miRNA as clinical markers is still challenging as it necessitates the quantification of targets molecules that are highly diluted in complex biofluids. Moreover, in many cases, it is necessary to measure more than one target to establish a diagnostic. Hence, the precise measurement of a set of nucleic acid biomarkers is the critical bottleneck which encourages the development of sensitive, quantitative and multiplex detection technologies.

The most used technique for sensitive detection of several proteins is the enzyme-linked immunosorbent assay (ELISA) and of nucleic acids, the polymerase chain reaction (PCR), for detecting DNA and the reverse transcription-quantitative polymerase chain reaction (RT-qPCR) for detecting RNA. All these techniques allow specific and sensitive detection but they have some drawbacks and need to be enhanced, particularly for a sensitive, quantitative and multiplex detection.

For example, currently the quantification of ADN through PCR involves the amplification of the target nucleic acid. This may induce carry-over contamination, which decreases the specificity and the sensitivity of the detection method.

Classical methods for the detection of RNA, particularly of miRNA include DNA (or LNA—locked-nucleic acid) microarrays ([1]-[5]) and PCR (polymerase chain reaction)-based technique (RT qPCR—reverse transcription quantitative PCR or RT dPCR-RT digital PCR, [6]-[9]). Microarrays offer great multiplexing capabilities (up to a few hundred targets). However, they suffer from poor sensitivity and poor specificity. Moreover, these methods do not allow an efficient quantification since the results are very dependent on experimental conditions. On the contrary, PCR-based methods demonstrate a good sensitivity but are limited in the best case to a few targets detected simultaneously. Additionally, they require a multistep procedure, data normalization and/or standard calibration, all of which introduce significant biases. Altogether, both techniques are not optimal for multiplex and quantitative detection and/or measurement of highly diluted targets.

The above-mentioned drawbacks can be overcome using a digital procedure. Digital techniques involve the partitioning of reaction sample into a large number of small compartments, for example microscopic droplets, inside of which the presence of individual molecule is reported, allowing direct counting. Digital techniques have several advantages, in particular: i) they are compatible with endpoint assays and do not require continuous monitoring of the reaction; ii) they provide absolute quantification without calibration standards; iii) they provide ultimate sensitivity; iv) they also improve the sensitivity and specificity of the assay especially in the case of rare targets within complex background.

For instance, digital PCR (dPCR ([19], [20])) is based on the isolation and on the amplification of individual targets in microcompartments and enables absolute quantification via digital single-molecule counting. Digital readout provides an absolute quantification without the need for assay calibration: it exploits the compartmentalization of single target molecules prior to signal amplification. As a result, only the compartments having received the target will amplify and display a positive (1) signal, while the others remain negative (0) (hence the qualifying term “digital”). By applying the Poisson law, one can calculate the exact concentration with a much higher precision than analog readouts, which are based on standard calibration.

However, digital PCR retains the drawbacks of the PCR amplification chemistry regarding tedious protocol optimization, sensitivity to inhibitors and biased quantification due to the reverse transcription step needed for nucleic acid detection, particularly for microRNA detection ([10]-[13]). Besides, the multiplexed format of digital assays has been poorly explored and is limited to a combination of 3-5 biomarkers due to crosstalk reactions between primers pairs and the restricted choice of spectrally-resolved fluorescence probes, respectively ([14]-[16]).

The sensitive detection of nucleic acids (DNA, RNA) is mostly based on target amplification by PCR-based techniques. Despite its very high sensitivity (down to the single molecule), this technique requires careful primer and probe design, optimization of the thermocycling protocol and expensive equipment. Additionally, the reverse transcription step needed for RNA detection is known to introduce quantification biases [13]. The detection of ribonucleic acids requires an additional reverse transcription step that converts the RNA sequence into a DNA strand usable for the PCR amplification, thus biasing the quantification.

Isothermal alternatives, some of them independent of a reverse transcription step, have been proposed, though the exponential amplification mechanism is prone to leaky reactions responsible for nonspecific amplification [17]. This intrinsic nonspecific reactivity prevents the use of these techniques for routine analysis. State-of-the-art protein and small analyte detection rely on ELISA (enzyme-linked immunosorbent assay) approaches using antigen/antibody recognition, which present a limited sensitivity that makes them suitable only for the detection of high concentration of analytes. It is to note that the coupling of the antibody recognition to nucleic acid amplification increases the specificity and sensitivity of the immuno-assay (immuno-PCR [18]).

Multiplex PCR and multiplex ELISA assays have been described and are generally based on orthogonal probes and/or amplification chemistries, which limit the number of targets to less than 10 (3-5 in general).

Another general problem with multiplex assay-based reactions such as PCR is that each target biomolecule to be detected requires an independent exponential reaction. Measuring, for example, 3 targets requires that the 3 amplification reactions are optimized to work in the same experimental conditions and without affecting each other. This use of multiple orthogonal amplification systems leads to complex experimental design and tedious optimization of the reaction conditions.

On the other hand, microarrays, based on the spatially-resolved capture of targets, allow to detect hundreds of markers at a time. Despite the high multiplexing capabilities of such arrays, the readout is analog, meaning that the average concentration of the sample is calculated from the number of bound targets, which limits the sensitivity. In addition, the measurement is poorly quantitative due to biased data normalization.

Therefore, while digital assay provides clear advantage in terms of usability and quantification, a multiplexed approach is required to enable better diagnostic. The multiplexed format of digital assays has been poorly explored and is limited to a combination of very few (3-6 biomarkers in the best cases [16]). For non-DNA targets, digital PCR suffers from the same problems associated to qPCR (i.e. conversion step, complex protocol design, thermocycling, enzyme's inhibition).

The purpose of the present invention is to provide a digital multiplex detection and/or quantification method allowing absolute quantification and higher sensitivity when detecting and/or quantifying multiple biomolecules, preferably used as biomarkers, such as biopolymers, particularly nucleic acids, but without the above cited drawbacks. The purpose is also to simplify the design of multiplex assay by using a single amplification mechanism for all targets.

### SUMMARY OF THE INVENTION

The inventors of the present invention have previously developed an analogous method for eliminating background amplification for the detection of nucleic acids which has been adapted to also detect multiple nucleic acids (WO2017140815). They surprisingly found that this analog multiplex method may be successfully digitalized in order to obtain a digital multiplex method allowing to increase the sensitivity of the detection of multiple biomolecules, preferably used as biomarkers, to quantify accurately these ones in a single measurement without necessity of performing several assays.

According to the first aspect, the present invention thus relates to a digital multiplex method for detecting and/or quantifying multiple biomolecules in a sample, comprising the following steps:
a) functionalizing a suspension of particles, preferably of microparticles with one or more oligonucleotides selected from a first oligonucleotide which is a conversion oligonucleotide (cT), a second oligonucleotide which is a reporting oligonucleotide (rT), a third oligonucleotide which is an amplification oligonucleotide (aT) and a fourth oligonucleotide which is a leak absorption oligonucleotide (pT);
b) adding to the particles functionalized in step a) barcodes allowing the discrimination of the particles targeting multiple biomolecules;
c) contacting the particles obtained in step b) with a tested sample to capture the multiple target biomolecules;
d) resuspending the particles having captured or not the target biomolecules in a common amplification mixture including a buffer, enzymes, deoxy-nucleoside triphosphate (dNTPs) and optionally oligonucleotides;
e) separating the particles in the suspension obtained in step d) from each other so that each particle can react independently;
f) incubating the particles at a constant temperature so that each target biomolecule triggers an amplification reaction which generates an amplification signal on the particle carrying the target, and
g) detecting and/or measuring the signals of the particles including the barcode signal and the amplification signal of each particle.

The method of the present invention allows the multiplex and digital detection of biomolecules targets, particularly of nucleic acid targets, hence giving access to the absolute concentration of multiple targets from a single assay. Besides, said method enables the direct quantification of the biomolecules targets of interest from complex media such as a biological sample since the particles can be washed after the capture step c). In fact, in standard assays such as PCR or other in-solution isothermal amplification chemistry, the mixture of sample and amplification mixture are usually polluted by the impurities present in the sample. Therefore, before quantifying, nucleic acid molecules have to be purified. With the digital multiplex method of the present invention, there is no need of additional extraction/purification step before contacting the particle with the tested sample, since the particles capture the target biomolecules irreversibly.

Another advantage of the digital multiplex detection and/or quantification method of the present invention is the possibility to adjust the dynamic range depending on the abundance of the target biomolecules in the sample. Indeed, the sensitivity of the method mostly depends on the ratio number of particles/number of targets (that is used to compute the concentration from the Poisson law). It is therefore possible to adjust the number of particles according to the target biomolecules (expected) abundancy which allows more accurate detection and controlling the quantity of used material in the method of the invention.

Moreover, when detecting and/or quantifying particularly RNAs, compared to the RT-PCR analog method, the advantage of the method of the invention is 1) the reverse transcription step, which may hamper the quantitatively of assay is not needed; 2) the high temperature and temperature cycling required for the PCR steps are not necessary; 3) the isothermal amplification is robust to many chemicals which may be found in crude samples and has been shown to inhibit the PCR reactions ([30] and [4]) the cross-contamination issues are avoided because the target biomolecule is not amplified.

Still another advantage of the digital multiplex method of the invention is that its design is simplified by using a single amplification mechanism for all multiple biomolecules.

The inventors also demonstrated that the method of the present invention may be used to detect biomolecules directly from any type of sample containing biomolecules, for example a blood sample and other biological fluids.

The specificity, the sensitivity, the simplicity and the rapidity of the digital multiplex method of the invention allow to use it in medical diagnosis methods, particularly for diagnosis of diseases such as cancer, neuronal diseases, cardiovascular diseases, inflammatory diseases, autoimmune diseases, diseases due to a viral or bacterial infection, skin diseases, skeletal muscle diseases, dental diseases and prenatal diseases.

According to the second aspect, the present invention also relates to an in vitro method for diagnosis of a disease selected from the group comprising or consisting of cancer, neuronal diseases, cardiovascular diseases, inflammatory diseases, autoimmune diseases, diseases due to a viral or bacterial infection, skin diseases, skeletal muscle diseases, dental diseases and prenatal diseases, said diagnostic method comprising the use of the multiplex digital method of the invention.

The specificity, the sensitivity, the simplicity and the rapidity of the digital multiplex method of the invention allow to also use it in agro diagnosis methods, particularly for diagnosis of diseases caused by biotic stress such as infectious and parasitic diseases, or caused by abiotic stress such as nutritional deficiencies or unfavorable environments.

According to the third aspect, the present invention also relates to an in vitro method for agro diagnosis of a disease selected from the group comprising:
diseases caused by biotic stress, preferably by infectious and/or parasitic origin, or
diseases caused by abiotic stress, preferably caused by nutritional deficiencies and/or unfavorable environment, said method comprising the use of the multiplex digital method of the invention.

For implementing the methods of the present invention, it is also provided a kit.

According to the fourth aspect, the present invention thus relates to a kit for detecting and/or quantifying multiple target biomolecules comprising:
a) a suspension of particles, preferably of microparticles functionalized with one or more oligonucleotides selected from a first oligonucleotide which is a converter oligonucleotide (cT), a second oligonucleotide which is a reporting oligonucleotide (rT), a third oligonucleotide which is an amplification oligonucleotide (aT) and a fourth oligonucleotide which is a leak absorption oligonucleotide (pT), to which particles are added barcodes allowing the discrimination of the particles targeting multiple biomolecules;
b) a mixture of enzymes, preferably selected from the group comprising polymerase, nicking enzyme or restriction enzyme, exonuclease and deoxy-nucleoside triphosphate (dNTPs) and optionally oligonucleotides, and
c) a separating agent.

### DETAILED DESCRIPTION

As indicated above, the present invention concerns the adaptation of recent background-free amplification chemistry to a supported format (i.e. at least a part of the chemical reaction is performed on a support) and digital readout, thus providing absolute quantification of multiple target biomolecules. Said digitalization allows the detection and/or the quantification of multiples biomolecules, preferably used as biomarkers with increased sensitivity and accuracy without necessity of performing several assays.

### Digital Multiplex Method for Detecting and/or Quantifying Multiple Biomolecules

According to the first aspect, the present invention thus relates to a digital multiplex method for detecting and/or quantifying multiple biomolecules in a sample, comprising the following steps:
a) functionalizing a suspension of particles, preferably of microparticles with one or more oligonucleotides selected from a first oligonucleotide which is a conversion oligonucleotide (cT), a second oligonucleotide which is a reporting oligonucleotide (rT), a third oligonucleotide which is an amplification oligonucleotide (aT) and a fourth oligonucleotide which is a leak absorption oligonucleotide (pT);
b) adding to the particles functionalized in step a) barcodes allowing the discrimination of the particles targeting multiple biomolecules;
c) contacting the particles obtained in step b) with a tested sample to capture the multiple target biomolecules;
d) resuspending the particles having captured or not the target biomolecules in a common amplification mixture including a buffer, enzymes, deoxy-nucleoside triphosphate (dNTPs) and optionally oligonucleotides;
e) separating the particles in the suspension obtained in step d) from each other so that each particle can react independently;
f) incubating the particles at a constant temperature so that each target biomolecule triggers an amplification reaction which generates an amplification signal on the particle carrying the target, and
g) detecting and/or measuring the signals of the particles including the barcode signal and the amplification signal of each particle.

The digital method according to the Invention allows to detect and/or quantify multiple biomolecules simultaneously, said biomolecules having the same or different structure and function. For that reason, the method of the present invention is called “multiplex” method.

In the context of the present invention, the term “biomolecule” relates to large macromolecules or biopolymers such as proteins, carbohydrates, lipids, and nucleic acids, as well as small molecules such as primary metabolites, secondary metabolites, and natural products. The biomolecules in the present invention are usually endogenous but may also be exogenous (for example, biopharmaceutical drugs).

According to one preferred embodiment of the digital multiplex method of the present invention, the biomolecules are selected from the group comprising proteins and nucleic acid. Said proteins being preferably enzymes.

In the context of the present invention the term “enzyme” designs proteins having catalytic function and which are able to catalyze the transformation of a molecular substrate. The groups of enzymes which may be detected and/or quantified by the digital multiplex method of the present invention are selected from nicking enzymes, restriction endonuclease, ADN N-glycosylase, AP-endonuclease, exonuclease ARN/ADN polymerase, ligase and methylase. Particularly, the digital multiplex method of the invention may be implemented for identifying and/or quantifying nicking enzymes and restriction endonuclease.

More preferably, the biomolecular targets of the present invention are nucleic acid such as DNAs, cDNAs, RNAs, mRNAs, microRNAs, even more preferably they are ribonucleic acids (RNAs).

In the context of the present invention, the term “nucleic acids” relates to biopolymers or small biomolecules composed of nucleotides, which are monomers made of three components: a 5-carbon sugar, a phosphate group and a nitrogenous base. If the sugar is a compound ribose, the polymer is RNA (ribonucleic acid); if the sugar is derived from ribose as deoxyribose, the polymer is DNA (deoxyribonucleic acid).

As mentioned above, the digital multiplex method of the present invention may be used for detecting and/or quantifying DNA molecules or complementary DNA (cDNA) which is coding molecule.

Even more preferably, the digital multiplex method of the present invention is used for detecting and/or quantifying ribonucleic acids (RNA) molecules selected from messenger RNA (mRNA), small interfering RNA (siRNA) and microRNA (miRNA).

According to the most preferred embodiment, the digital multiplex method of the present invention may be used to detect and/or quantify microRNA molecules.

In the context of the present invention, the term “microRNA” designs endogenous short non-coding RNA strands, with a length around 22 nucleotides involved in the post-transcriptional regulation of gene expression.

In order to implement the method of the present invention it is necessary in the first step (step a)) to functionalize a suspension of particles, preferably of microparticles with one or more oligonucleotides selected from a first oligonucleotide which is a conversion oligonucleotide (cT), a second oligonucleotide which is a reporting oligonucleotide (rT), a third oligonucleotide which is an amplification oligonucleotide (aT) and a fourth oligonucleotide which is a leak absorption oligonucleotide (pT).

According to one embodiment, the particles may be functionalized with two oligonucleotides: a conversion oligonucleotide (cT) and a reporting oligonucleotide (rT).

According to another embodiment, the particles are functionalized with three oligonucleotides: a conversion oligonucleotide (cT), an amplification oligonucleotide (aT) and a leak absorption oligonucleotide (pT); a conversion oligonucleotide (cT), reporting oligonucleotide (rT) and an amplification oligonucleotide (aT); a conversion oligonucleotide (cT), reporting oligonucleotide (rT) and a leak absorption oligonucleotide (pT).

According to still another embodiment, the particles are functionalized with four oligonucleotides: a conversion oligonucleotide (cT), a reporting oligonucleotide (rT), an amplification oligonucleotide (aT) and a leak absorption oligonucleotide (pT).

In the context of the present invention, the term “conversion oligonucleotide” or “target specific conversion oligonucleotide” or “conversion template” or “cT” relates to an oligonucleotide which converts the target biomolecule to a universal short DNA oligonucleotide (or also called herein “signal” or “signal sequence”). The conversion template may or may not be protected against degradation.

According to one preferred embodiment of the method of the present invention, the conversion template may be designed in a specific manner, depending of the biomolecule to be detected. For example, when a given microRNA is detected, the most 3′ portion of the conversion template is complementary or partially complementary to this microRNA target. The 5′ portion corresponds to the complementary sequence of the output (signal) sequence. In between these two elements is introduced the nicking recognition/cutting site so that the strand polymerized along the template, using the microRNA as a primer can be nicked to release the output sequence. In the case of the nicking enzyme Nt.BstNBI (used in the examples described hereafter) this sequence may be 5′-NNNNGACTC-3′ (where N is one of the four canonical DNA bases).

Optionally, for designing the conversion template, a spacer sequence may be included between the biomolecule binding site and the nicking recognition site. The spacer sequence comprises an oligodeoxyribonucleotide sequence between 1 and 20 nucleotides, preferably between 4-12 nucleotides, more preferably between 5-10 nucleotides. In one embodiment, the spacer sequence in composed of a sequence missing at least one of the 4 nucleotides (e.g. T, A, C but not G).

Particularly, the spacer is poly(T) spacer. In the context of the present invention, the term “poly(T) spacer” designs a poly(T) homopolymer spacer with a length comprised between 1 and 20 nucleotides, preferably between 4-12 nucleotides, more preferably between 5-10 nucleotides.

According to particularly preferred embodiment, the conversion template comprises a poly(T) spacer in the target biomolecule binding sequence (for example in miRNA binding sequence) and the nicking enzyme (for example Nt.BstNBI) recognition site, thus increasing the number of DNA nucleotides on the primer obtained after extension and nicking.

This modified design of the conversion template allows to significantly reduce or even eliminate the unspecific amplification (also called background amplification) and thus increasing the sensitivity and the reliability of the method of the invention.

Examples of different designs of the conversion template are given below:

| | |
|---|---|
| | (SEQ ID NO: 75) |
| 16-5toBc T5 T7 | 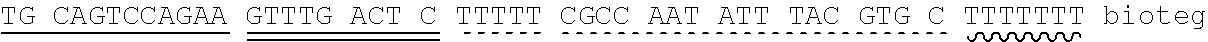 |
| biot | |
| | (SEQ ID NO: 76) |
| 10atoBc T5 T7 | 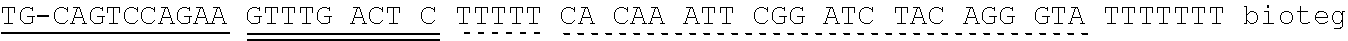 |
| biot | |
| | (SEQ ID NO: 77) |
| 203atoBc | 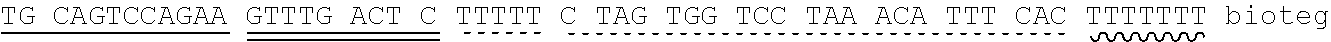 |
| T5 T7 biot | |
| | (SEQ ID NO: 78) |
| Lin4toBc | 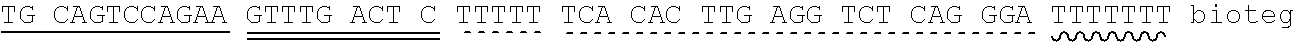 |
| T5 T7 biot | |
| | (SEQ ID NO: 79) |
| Let7atoBc | 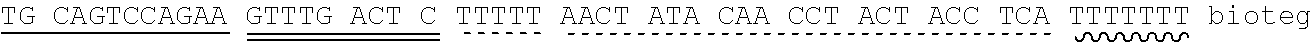 |
| T5 T7 biot | |
| | (SEQ ID NO: 80) |
| 21toBc T5 T7 | 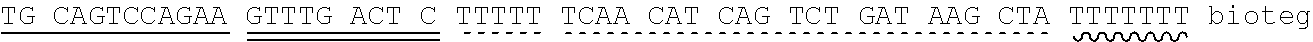 |
| biot | |
| | (SEQ ID NO: 81) |
| 21toBc T10 T7 | 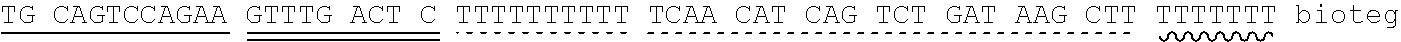 |
| biot | |
| | (SEQ ID NO: 82) |
| 21toBc T15 T7 | 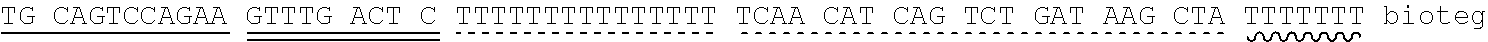 |
| biot | |

In these sequences, the nucleotides underlined by:
a bold line

(TG-CAGTCCAGAA),

corresponds to a complementary output sequence;
a double line

(GTTTG ACT C),

corresponds to NtBstNBI recognition site;
not full line [symbol] , corresponds to the spacers;
dotted line [symbol] , corresponds to the biomolecule binding site, particularly to the microRNA binding site, and
wavy line [symbol] , corresponds to a linker.

In the context of the present invention, the term “reporting oligonucleotide” or “reporting probe” or “reporting template” or “rT” relates to an oligonucleotide which translates the presence of the trigger (signal sequence) sequence into a detectable signal. Such detectable signal is for example a fluorescent signal, electrochemical signal, particles aggregation, colorimetric signal, preferably a fluorescent signal. Thus, the reporting probe is preferably a fluorescent probe.

In one embodiment, the reporting probe is a self-complementary structure modified at both extremities by a fluorophore and/or a quencher. As used herein, the term “self-complementary” means that two different parts of the same molecules can hybridize to each other due to base complementary (A-T and G-C). In the case of the invention, the two extremities of the single strand probe (a few nucleotides on the 3′ and 5′ part) can hybridize to each other and induce the quenching of the fluorophore by the quencher.

The reporting probe may also comprise a loop which includes a nicking recognition site.

Compared to PCR probes (used for detecting) used in the prior art which must be designed (sequence) and optimized (length) for each target, the reporting probes used in the digital multiplex method of the invention are modular, meaning that they can be used for any target through signal conversion by the proper bistable module (autocatalytic template+pseudotemplate described below).

In the context of the present invention, the term “amplification oligonucleotide” or “autocatalytic template” or “aT” designs an oligonucleotide which is able to exponentially amplify the trigger (signal sequence). The examples of oligonucleotides which may function as amplification oligonucleotides are given in table 1 below.

According to one embodiment of the digital method of the invention, the amplification oligonucleotide includes a partial repeat structure containing a nicking enzyme recognition site, and the leak-absorption oligonucleotide is able to bind, extend, deactivate and slowly release the products of polymerization along the amplification oligonucleotide, thereby inducing a threshold.

According to one embodiment of the digital multiplex method of the invention, a 3′ end of the amplification oligonucleotide has a reduced affinity for an amplified sequence.

In the context of the present invention, the term “leak absorption oligonucleotide” or “pseudotemplate” or “pT” relates to the oligonucleotide which binds the amplified sequence more strongly than the autocatalytic template and drives the addition of few nucleotides at the 3′ of the amplified sequence, thus deactivating it for further priming on the autocatalytic template (because the 3′ end of the deactivated amplified sequence is now mismatched on the autocatalytic template). The leak-absorption oligonucleotide drives the deactivation of triggers (signal sequences) synthetized by leaky reactions and therefore allows to avoid nonspecific amplification, also called herein background amplification (i.e. the amplification occurring in absence of the amplified signal sequence). The pseudotemplate needs to be protected against degradation by the exonuclease. This is done using a few phosphorothioates modifications at its 5′ end (or other exonuclease-blocking possibilities such as biotin-streptavidin modifications or modified bases). Examples of leak absorption oligonucleotide are given in table 1 below.

In still another embodiment, a 3′ end of the leak absorption oligonucleotide is complementary to the sequence amplified by the amplification oligonucleotide, and a 5′ end of the leak absorption oligonucleotide serves as a template to add a deactivating tail to the amplified sequence.

In one embodiment for implementing the present digital multiplex method, the concentrations of the amplification and the leak absorption oligonucleotides are selected so that a reaction of the amplification oligonucleotide is faster than the reaction of the leak absorption oligonucleotide at high concentration of the amplified sequence but the reaction on the leak absorption oligonucleotide is faster than the reaction of the amplification oligonucleotide at low concentration of the amplified sequence, thereby effectively eliminating amplification unless the stimulus threshold is crossed.

Each oligonucleotide used in the digital multiplex method of the present invention has specific function finally allowing the conversion of the target biomolecules and the amplification of the obtained signal without inducing at the same time a background amplification (i.e. the amplification occurring in absence of the amplified signal sequence).

Examples of different oligonucleotides (templates) used in the digital multiplex method of the invention are given in table 1 below and in the Examples section of the application.

TABLE 1
Examples of oligonucleotides (templates).

| SEQ ID | Name of the | | |
|---|---|---|---|
| NO: | sequence | Nucleotides Sequence | Function |
| SEQ ID | CBe12PS3: | C*G*A*TCCTGAATG-CGATCCTGAATG-p | autocatalytic |
| NO: 1 | | | template |
| SEQ ID | CBe12-1PS3 | C*G*A*TCCTGAATG-CGATCCTGAAT-p | autocatalytic |
| NO: 2 | | | template |
| SEQ ID | CBe12-2PS3: | C*G*A*TCCTGAATG-CGATCCTGAA-p | autocatalytic |
| NO: 3 | | | template |
| SEQ ID | ptBe12T5SP: | T*T*T*T*T-CGATCCTGAATG-p | pseudotemplate |
| NO: 4 | | | |
| SEQ ID | CBa12-1PS4: | C*T*C*G*TCAGAATGCTCGTCAGAAT-p | autocatalytic |
| NO: 5 | | | template |
| SEQ ID | ptBa12A4SP: | A*A*A*ACTCGTCAGAATG-p | pseudotemplate |
| NO: 6 | | | |
| SEQ ID | ptBa12T5SP | T*T*T*T*T-CTCGTCAGAATG-p | pseudotemplate |
| NO: 7 | | | |
| SEQ ID | ptBa12T4S3P: | T*T*T*T-CTCGTCAGAATG-p | pseudotemplate |
| NO: 8 | | | |
| SEQ ID | ptBa12T3S3P: | T*T*T*-CTCGTCAGAATG-p | pseudotemplate |
| NO: 9 | | | |
| SEQ ID | ptBa12T2S3P: | T*T*C*TCGTCAGAATG-p | pseudotemplate |
| NO: 10 | | | |
| SEQ ID | ptBa12T1S3P: | T*-C*T*CGTCAGAATG-p | pseudotemplate |
| NO: 11 | | | |
| SEQ ID | CBa12-2PS4: | C*T*C*G*TCAGAATG-CTCGTCAGAA-p | autocatalytic |
| NO: 12 | | | template |
| SEQ ID | CBa12PS4: | C*T*C*G*TCAGAATG-CTCGTCAGAATG-p | autocatalytic |
| NO: 13 | | | template |
| SEQ ID | CBa12-1PS4: | C*T*C*GTCAGAATG-CTCGTCAGAAT-p | autocatalytic |
| NO: 14 | | | template |
| SEQ ID | CBa12-3PS4: | C*T*C*G*TCAGAATG-CTCGTCAGA----p | autocatalytic |
| NO: 15 | | | template |
| SEQ ID | pTk12T5S4P: | T*T*T*T*T-CAATGACUCCTG-p | pseudotemplate |
| NO: 16 | | | |
| SEQ ID | ApTk12A1SUP: | A*C*A*ATGACUCCTG-A-p | pseudotemplate |
| NO: 17 | | | |
| SEQ ID | ApTk12A2SUP: | A*A*C*AATGACUCCTG-A-p | pseudotemplate |
| NO: 18 | | | |
| SEQ ID | ApTk12A3PS: | A*A*A*-C*AATGACUCCTG-A-p | pseudotemplate |
| NO: 19 | | | |
| SEQ ID | ApTk12A4SUP: | A*A*A*ACAATGACUCCTGA-p | pseudotemplate |
| NO: 20 | | | |
| SEQ ID | ApTk12A5SUP: | A*A*A*AACAATGACUCCTG A-p | pseudotemplate |
| NO: 21 | | | |
| SEQ ID | ApTk12A6SUP: | A*A*A*AAACAATGACUCCTGA-p | pseudotemplate |
| NO: 22 | | | |
| SEQ ID | Ck12-2PS4bioteg | bioteg*C*A*A* TGA CUC CTG CAA TGA CTC | Autocatalytic |
| NO: 23 | | C p | template |
| SEQ ID | Cba12-2biot3 | C*T*C*G*TCAGAATG CTCGTCAGAA bioteg | autocatalytic |
| NO: 24 | | | template |
| SEQ ID | ptBa12A5SP | A*A*A*A*A CTC GTC AGA ATG p | pseudotemplate |
| NO: 25 | | | |
| SEQ ID | Cbe12-2S4P | C*G*A*T*CCTGAATGCGATCCTGAA p | autocatalytic |
| NO: 26 | | | template |
| SEQ ID | ApTBe12A3S3P | A*A*A*CGATCCTGAATGAp | pseudotemplate |
| NO: 27 | | | |
| SEQ ID | CBe12-2noPS3 | C*G*A*TCCTGAATGCGATCCTGAA | autocatalytic |
| NO: 28 | | | template |
| SEQ ID | Ck12- | Bioteg CAA TGA CUC CTG CAA TGA CTC C p | autocatalytic |
| NO: 29 | 2S4noUbioteg | | template |
| SEQ ID | ApTk12A5S3P | A*A*A*AACAATGACUCCTGA p | pseudotemplate |
| NO: 30 | | | |
| SEQ ID | CBe12-2AULP | C*G*A*TCCTGAATGCGATCCTGA | autocatalytic |
| NO: 31 | | | template |
| SEQ ID | D21tof5TBe12S3P | C*G*A*TCCTGAAAGCGAAGTTTGACTC | conversion |
| NO: 32 | | ATCAACATCAGTCTGATAAGCTA p | template |
| SEQ ID | CBe12-3noPS3 | C*G*A*TCCTGAATGCGATCCTGA | autocatalytic |
| NO: 33 | | | template |
| SEQ ID | D21tofBe12S0P | CGATCCTGAATG TCA ACA TCA GTC TGA TAA | conversion |
| NO: 34 | | GCT A p | template |
| SEQ ID | CBe12-2SPCy355 | Cy3.5 | autocatalytic |
| NO: 35 | | *C*G*ATCCTGAATGCGATCCATCCTGAA p | template |
| SEQ ID | CBc12SPBMN35 | BMN3*C*A*G*TCCAGAATGCAGTCCAGAA p | autocatalytic |
| NO: 36 | | | template |
| SEQ ID | pTBc12T5SP | T*T*T*T*TCAGTCCAGAATG p | pseudotemplate |
| NO: 37 | | | |
| SEQ ID | 92atoF5TBe12PS0 | CGA TCC TGA AAG CGA AG T TTG ACT CAA | conversion |
| NO: 38 | | GCA TTG CAA CCG ATC CCA ACC p | template |
| SEQ ID | Let7atof5TBa12S0P | CTC GTC AGA AAG CGA AGT TTG ACT CAA | conversion |
| NO: 39 | | ACT ATA CAA CCT ACT ACC TCA p | template |
| SEQ ID | CBa12-2AULP | C*T*C*GTCAGAATGCTCGTCAGAAAGCGAAG | autocatalytic |
| NO: 40 | | C p | template |
| SEQ ID | ApTBa12A3S3P | A*A*A*CTCGTCAGAATGA | pseudotemplate |
| NO: 41 | | | |
| SEQ ID | RPBe-Cy5 | bioteg TTT TG DDQII CAT TCA ATT TTC GAT | reporting probe |
| NO: 42 | | CCT GAA TG Cy5 | |
| SEQ ID | CBe12-1S4bioteg | bioteg*C*G*A*TCCTGAATGCGATCCTGAAT p | autocatalytic |
| NO: 43 | | | template |
| SEQ ID | Cba125bioteg | bioteg*C*T*C*GTCAGAATGCTCGTCAGAATG p | autocatalytic |
| NO: 44 | | | template |
| SEQ ID | RPBa-Hex | biotin TTTTG BMNQ530 AATTCTATTTT CTC | reporting probe |
| NO: 45 | | GTC AGA ATT Hex | |
| SEQ ID | Cba12-3noPS3 | C*T*C*GTCAGAATG CTCGTCAGA | autocatalytic |
| NO: 46 | | | template |
| SEQ ID | RPBe-Cy5(2) | Cy5 *T*T*CAGGTTTTCGATCCTGAA BHQ2 | reporting probe |
| NO: 47 | | | |
| SEQ ID | RPBe-Cy5(3) | Cy5 *A*T*TCAGAATGCGATCCTGAAT BHQ2 | reporting probe |
| NO: 48 | | | |
| SEQ ID | ptBa12A6biot | biotin*A*A*AAAACTCGTCAGAATG p | pseudo-template |
| NO: 49 | | | |
| SEQ ID | 92atof1Be12-3+3 | ATGCGATCCTGACGTTTGACTCAA GCA TTG | conversion |
| NO: 50 | | CAA CCG ATC CCA ACC | template |
| SEQ ID | Let7atof1Ba12- | ATGCTCGTCAGA CGT TTG ACT CAA ACT ATA | conversion |
| NO: 51 | 3+3 | CAA CCT ACT ACC TCA | template |
| SEQ ID | CBc12-3noPS3 | C*A*G*TCCAGAATGCAGTCCAGA | autocatalytic |
| NO: 52 | | | template |
| SEQ ID | RPBc-FAM | FAM*T*T*CTGG TTTTCAGTCCAGAA BHQ1 | reporting probe |
| NO: 53 | | | |
| SEQ ID | Atoα | C*A*G*T*CCAGAATGCAGTCCAGAA p | autocatalytic |
| NO: 54 | | | template |
| SEQ ID | pTα | T*T*T*T*TCAGTCCAGAATG p | pseudo template |
| NO: 55 | | | |
| SEQ ID | rTα | Atto633*A*T*TCTGAATGCAGTCCAGAAT | reporting template |
| NO: 56 | | BHQ2 | |
| SEQ ID | Let7atoα | TGCAGTCCAGAAGTTTGACTCAAACTATACAA | conversion |
| NO: 57 | | CCTACTACCTCA p | template |
| SEQ ID | Let7ctoα | TGCAGTCCAGAAGTTTGACTCAAACCATACAA | conversion |
| NO: 58 | | CCTACTACCTCA p | template |
| SEQ ID | mir39toα | TGCAGTCCAGAAGTTTGACTCACAAGCTGATT | conversion |
| NO: 59 | | TACACCC p | template |

In the table 1 above, biotin and bioteg refer to biotinylated synthons, respectively using aminoethoxy-ethoxyethanol linker and the longer triethylene glycol linker. “*” denotes a phosphorothioate backbone modification and “p” designates a 3′ phosphate modification. In SEQ ID Nos: 16 to 23 and 30, thymidine (T) is replaced by deoxyuridine (U) in order to avoid the nicking of the product of polymerization of the signal on the template. Atto633, FAM, Cy5, HEC, BMN3, Cy3.5 are fluorophores, BHQ2, BHQ1 and BMNQ530 are quenchers.

On the basis of the sequences listed above and the examples described herein, various combinations of templates may be used for implementing the digital multiplex method of the invention. The person skilled in the art, starting from the present description and general knowledge concerning nucleotide design will be able to determine other templates and other combination in order to implement the method of the invention for any target biomolecule of interest.

Particularly, the selection of the sequence of templates depends on experimental parameters like the working temperature, the speed and specificity of the used enzymes, particularly of the nickases. Preferably, amplification templates that contains the Nb.Bsml nickase site and/or templates that work with Nt.BstNBI or Nb.BssSI may be used. However, any other nicking enzyme may be used, including Nt.BspQI, Nt.CviPII, Nb.BsrDI, Nb.BtsI, Nt.AlwI, Nb.BbvCI, Nt.BbvCI and Nt.BsmAI, among others. More than one nickase and more than one nickase recognition site can be used in the same mixture.

In the context of the present invention, the functionalization of the particle means fixing or attaching any one of the oligonucleotides to the particle.

The oligonucleotides used in the digital method of the invention may be thus modified in order to be fixed on the particle surface. For example, in the method of the present invention a biotin-avidin linkage may be used to attach the oligonucleotides to the particle, but many other grafting chemistry could be used to attach the oligonucleotide to the particle, including but not limited to amino coupling, disulfide bonds, carboxyl coupling, self-assembled monolayer, other thiol-reactive chemistry, click chemistry, dual-biotin-avidin linkage, nucleic linker-mediated hybridization and any covalent ligation and non-covalent immobilization chemistry.

In one preferred embodiment of the method of the invention, the modified oligonucleotides fixed on the particle surface are conversion oligonucleotide (cT) and/or reporting oligonucleotide (rT).

According to one embodiment, instead of being directly attached to the surface of the particle, some oligonucleotides could be attached to the free end of the attaching means (or linker) using a nucleic acid sequence or non-polymerizable linker, such as PEG linker or aliphatic linker (which may be also called “a spacer”). Such spacer may be for example nucleic acid spacer (modified or not polynucleotides) or non-polymerizable spacers, such as polyethylene glycol spacers or aliphatic spacers.

In one embodiment of the digital multiplex method of the invention, the functionalization of the suspension of particles, preferably of microparticles in step a) is performed with the conversion oligonucleotide (cT) and with the reporting oligonucleotide (rT) only.

In this embodiment, only the conversion oligonucleotide (cT) and the reporting oligonucleotide (rT) are fixed on the particles before contacting this particle with barcode and with the tested sample. The third oligonucleotide (aT) and fourth oligonucleotide (pT) are added later, in the amplification mixture in step d). In this embodiment, the amplification mixture in step d) also comprises oligonucleotides. In preferred embodiment a biotin-(strept)avidin linkage is used to attach the first and the second oligonucleotides to the particle, but as indicated above, many other grafting chemistries could be used to attach these oligonucleotides. Moreover, a spacer may also be used.

The particles used in the digital multiplex method of the present invention are preferably microparticles.

According to one embodiment, these particles are porous, i.e. they are made from porous material.

According to another embodiment, the particles are made from hydrogel. Such hydrogel particle may be performed for example by the protocol described by Xu et al. (2005).

According to still another embodiment, the particles are non-porous solid particles.

The size of the particles used in the method of the present invention is comprised between 10 nm and 500 μm, preferably between 100 nm and 100 μm and more preferably, between 500 nm and 10 μm. In one preferred embodiment, said particles are 1 μm streptavidin-coated particles. Such particles may be purchased for example from Invitrogen.

According to another embodiment of the digital multiplex method of the present invention, the conversion oligonucleotide and the reporting oligonucleotide may be biotinylated in order to be attached to a avidin or streptavidin modified particle.

According to one embodiment, the digital multiplex method of the invention may comprise, prior to step a) of functionalization of the particles, a step of washing the particles with a buffer and then resuspending the washed particles in the same buffer in order e.g. to remove the preservatives contained in storage buffer.

A washing may be also performed after the step a) of functionalization. However, the advantage of the multiplex method of the present invention is that such washing is not necessary. The avoiding of washing step allows to perform more rapidly and accurately the detection and/or the quantification of the target biomolecule.

In step b) of the digital method of the present invention, to the functionalized particles in step a) are added barcodes allowing the discrimination of the particles targeting multiple biomolecules. Thus, each particle batch is barcoded according to the biomolecule it targets.

In the context of the present invention, the term “barcode” thus relates to a label that is attached or associated with the particles and allows the discrimination between the different populations of particles that are used to detect different target biomolecules of interest.

A number of strategies have been developed to barcode particles as cited for example in the references [21]-[25]. According to one embodiment, a fluorescent dye can be grafted (covalently or not), or directly integrated to the particle by mixing it to the monomer mixture, as it can be done with quantum dots. According to another embodiment, a Raman spectroscopy labels, a photonic crystal and/or a rare-earth ions integration may be used for barcoding the particles.

Other means for discriminating the particle populations in the digital multiplex method of the present invention may comprise using particles having different shape, size or granulometry.

In one preferred embodiment, the barcode is a fluorescent barcode. Preferably, said fluorescent barcode is a combination of fluorescent molecules at different grafting density giving each particle type a discriminable identity when excited with a fluorescence recording apparatus.

Particularly, each particles population is barcoded with a fluorescent dye by co-grafting a biotin labeled fluorescent oligonucleotide, so that they can be differentiated using their fluorescent properties.

In one preferred embodiment of the digital method of the present invention, step a) of functionalization of the particles and step b) of barcoding as performed concomitantly, i.e. the barcodes are added at the same time as on or more of four oligonucleotides.

Once the particles have being functionalized and barcoded in steps a) and b) respectively or concomitantly, they are contacted with a tested sample to capture the different target biomolecules in step c) of the digital multiplex method of the present invention.

As used herein, the terms “target biomolecule” or “biomolecule target” relates to the biomolecules as defined above which is detected and/or quantifying by the method of the invention.

These target biomolecules are present in a sample. Said sample may be of any type. For example, said sample may be obtained from a tested subject, said subject being an animal, preferably a mammal and more preferably a human. Such sample may be also called a biological sample.

In the context of the present invention, the term “biological sample” relates to a solid or fluid biological material obtained from a living organism. Solid biological samples may be such as cells, part of tissue (biopsy), whole tissue or organ.

Preferably, the sample used in the method of the present invention is a fluid sample.

In the context of the present invention, the terms “sample of biological fluid” or “fluid sample” relate to any sample obtaining from bio-organic fluids produced by a life organism. The biological fluids are selected from the group comprising extracellular fluids, intravascular fluids, interstitial fluids, lymphatic fluids and transcellular fluids.

Particularly, the sample of biological fluid is selected from the group comprising blood and blood components, urine, saliva, tears, sweat, etc.

In more preferred embodiment the sample of biological fluid is a sample of blood or a sample of blood components. “Sample of blood” or “sample of blood components” means the total blood or one of its components selected especially from the red cells fraction, white cells fraction, platelets, plasma or serum.

In another embodiment of the present invention, the sample may be obtained from a non-living organism. For example, said sample may be obtained from air, water, soil, alimentary product etc. The type of the sample depends of the application of the digital multiplex method of the invention. According to the present invention, the said sample contains or is susceptible to contain biomolecules.

In step c), via a hybridization with the conversion oligonucleotide (cT), the target biomolecules are randomly captured by the cognate particle following a Poisson distribution. For that, the number of particles has to be such that the Poissonian capture of a number of target biomolecules gives a fraction of particles having captured at least one target that is less than 100% of the total number of particles. Preferably this number is less than 95%.

In one embodiment, the particles are mixed with the tested sample in a reaction buffer. After incubation, the particles are pelleted and resuspended in a storage buffer at a concentration comprised between 10⁶ and 10¹² particles/mL, preferably between 10′ and 10¹¹ particles/mL and more preferably 10⁸ and 10¹⁰ particles/mL. In the most preferred embodiment, the concentration of the particles is 10⁹ particles/mL. According to one embodiment of the method of the present invention, after step c), the particles having captured or not the target biomolecules may be washed in washing buffer before the next step d) of resuspending the particles in the amplification mixture.

According to preferred embodiment, following the capture of the target biomolecule on the particles, the particles may be washed. Particularly, they are washed when a polymerase such as Klenow polymerase is used in step d). In this last case the particles are resuspended several times in washing buffer (for example, between 2 and 10 times, preferably, between 3 and 7 times and more preferably, 4 or 5 times).

In step d) of the digital multiplex method of the present invention, the particles capturing the target biomolecules are resuspended in an amplification mixture including a buffer, enzymes, deoxy-nucleoside triphosphate (dNTPs) and optionally oligonucleotides.

In the context of the present invention, the term “amplification mixture” relates to a reactional mixture containing agents allowing amplifying the target sequences. Particularly, these agents are selected from a buffer, enzymes, deoxy-nucleoside triphosphate (dNTPs) and optionally oligonucleotides as defined below.

According to one embodiment of the digital multiplex method of the invention, the enzymes used in step d) are selected from the group comprising polymerase, nicking enzyme or restriction enzyme and exonuclease. The polymerase, the nicking enzyme and the restriction enzyme can drive the isothermal amplification and the exonuclease can avoid saturation of the system.

Particularly, the polymerase used in the digital multiplex method of the present invention is selected from the group comprising Bst 2.0 DNA polymerase, Bst large fragment DNA polymerase, Klenow fragment (3′->5′exo-) (also cited herein as Klenow polymerase), Phi29 DNA polymerase, Vent(exo-) DNA polymerase, more particularly, the polymerase is Vent(exo-) DNA polymerase (purchased from New England Biolabs). A mixture of two or more polymerases can also be used.

According to a preferred embodiment polymerases Vent(exo-) DNA polymerase and Klenow fragment (3′->5′exo-) are used together. Particularly, in this case, Klenow fragment (3′->5′exo) allows to improve the capturing of the target biomolecule on the surface of the particle before the encapsulation of the particle. Vent(exo-) DNA polymerase is then used in the amplification reaction.

The nicking enzyme is selected from the group comprising Nb.BbvCI, Nb.BstI, Nb.BssSI, Nb.BsrDI particularly, the nicking enzyme is Nb.Bsml and/or Nt.BstNBI (purchased from New England Biolabs (NEB)). A mixture of two or more nickases can also be used.

According to one embodiment of the method of the invention, the nicking enzymes may be replaced by restriction enzymes. Contrary to the nicking enzymes which cut only one strand, the restriction enzymes cut the double strand. Thus, when using restriction enzymes instead to nicking enzymes, it is necessary to protect the oligonucleotides used in the method of the invention. This protection may be performed for example by performing chemical modification of the oligonucleotides. Such modifications comprise phosphorothioate backbone modification, locked nucleic acid sugar modification, peptide nucleic acid modification or replacement of one nucleobase from another, for example methylation of the nucleobases (e.g. replacement of guanine by O⁶-methyl guanine or cytosine by methyl cytosine). The examples of such modifications which may be also used in the method of the present invention are disclosed by Loenen et al. (2014)

The exonuclease used in the digital method of the invention is selected for example from RecJf, Exonuclease I, Exonuclease VII, particularly, the exonuclease is ttRecJ exonuclease obtained following the protocol described by Yamagata (Yamagata et al., 2001).

The reaction buffer used for the mixture of enzymes and oligonucleotides is adapted to the selected oligonucleotides templates. The skilled artisan would be able to adapt conventional buffers to particular molecular design. The experimental part of the present application also gives examples of such buffers. For example in preferred embodiment in step c) and d) of the method of the invention, the reaction buffer comprises 20 mM Tris HCl pH 7.9, 10 mM (NH₄)₂SO₄, 40 mM KCl, 10 mM NaCl, 10 mM MgSO₄, 25 μM each dNTP, 0.1% (w/v) Synperonic F 104, 2 μM netropsin and 200 μg/mL BSA.

In one embodiment, the capturing step c) and the resuspending step d) can be performed at the same time, i.e. the particles and the sample containing the target biomolecules are injected directly in the amplification mixture. In this case, the washing step may be avoided. In general manner, this embodiment is performed when the tested sample is not toxic or not obstructing for the amplification reaction. For example, when the sample contain purified extracts of microRNA.

After step d) of resuspending the particles having captured or not the target biomolecules, a step e) of separating the particles in the suspension from each other so that each particle can react independently is performed.

According to one embodiment, the separation of particles may be obtained by different means using microchambers arrays, droplets microfluidics or particle dispersion in an immiscible fluid. For example, the separation step is performed in microdroplet (such as for instance QX200 system from Biorad or Naica System from Stilla technologies). Particularly, the separation is performed into millions of water-in-oil droplets made by combining two immiscible liquids: the sample aqueous phase and the continuous hydrophobic phase such as undecane-1-ol, silicon oil, mineral oil, more preferably perfluorinated oils because of their high immiscibility with water, biocompatibility, low viscosity, transparency and compatibility with PDMS-made devices.

According to one preferred embodiment, the separation is performed by generating a water-in-oil emulsion with aqueous droplets containing the particles suspended in the reaction mixture.

According to one embodiment, the droplets used in the digital method of the invention have a size comprised between 0.001 pL and 100 pL, preferably between 0.1 pL and 10 pL and more preferably between 0.5 and 5 pL.

In another embodiment, the separation in step e) may be performed by using a microfluidic chip which allows Poissonian distribution of the particles in the drops of the used emulsion.

The concentration of the particles in the amplification mixture prior to step e) of separation when the separation is performed in microfluidic droplets is selected such that on average only a small number of particles get encapsulated in each droplet. Preferably, this number is smaller than one.

Other microfluidic method allowing to obtain Poisson law distribution and to obtain a small number of particles or single particle per drop of emulsion and minimum of empty drops are selected from acoustic focus, inertial focus, “close-packed ordering”. These methods are known to skilled artisan who can adapt the parameters for their implementation to the digital multiplex method of the invention.

Moreover, for separating particles in step e) a specific device such as microchambers device (such as for instance SlipChip or QuantStudio 3D from Thermofisher Scientific) could be used wherein the particles are distributed in the microchambers which separate them from each other.

According to another embodiment, the particles are physically isolated by dispersion in an immiscible fluid or chemically by closing the pores of the particles (for instance using layer by layer deposition or polymer cross-linking or media jellification). In this embodiment, the amplification reaction is performed in the pores of the particle.

In another embodiment, the separating step may be performed with non-porous particles. In this case, the amplification reaction is performed on the particle which is surrounded by liquid microlayer allowing to separate said particle from the other particles. This allows to limit the signal diffusion between the particles.

As used herein, the term “digital multiplex method” designs a detection and/or quantification method, wherein a multiplicity of targets are randomly captured by a collection of functionalized and barcoded particles, each particle type being specific for a target type and each target type being captured by the particles following a Poissonian distribution. A signal amplification reaction is subsequently carried out in or around (in the liquid microlayer) each particle so that only the particles having captured at least one target exhibit a positive signal. This allows a direct counting of discrete events for each particle population and therefore the absolute quantification of the associated target biomolecules.

The main advantage of the digital multiplex method of the present invention compared to the PCR digital multiplex method (wherein the primer and the probes are specific for each target biomolecule) for example is due on the fact that the multiple target biomolecules captured by the particles are not directly detected but are converted into a common signal sequence and subsequently into a common measurable signal. This allows to avoid the sensitivity and specificity issues caused by the direct detection of the target biomolecules as well as complex design and implementation of orthogonal amplification systems.

The digital multiplex method of the present invention, also comprises a step f) of incubating the particles at a constant temperature so that each target biomolecule triggers an amplification reaction and a reporting signal.

As used herein, the term “signal” or “signal sequence” relates to nucleic acid sequence, preferably a single strand DNA which is obtained by converting the sequence of the targeted biomolecule, preferably nucleic acid molecule, more preferably, a microRNA into a sequence which may be amplified.

The converted signal sequence is thus amplified in step f) of the digital multiplex method of the invention.

According to one embodiment, the amplification in step f) is performed at a constant working temperature ranging from 35 to 60° C., more preferably from 37 to 55° C. and even more preferably from 45 to 50° C.

In one embodiment, the digital multiplex method of the invention comprises a step f1) of recovering the particles. In one preferred embodiment, the recovering is performed by breaking the compartments containing the particles. In such a way, the particles are recovered in a solution, preferably, in an aqueous medium. The recovering of particles is performed when the particles have been separated from each other by emulsification, i.e in an unmissable liquid, preferably, in water-in-oil emulsion. The breaking of the compartments in step f1) may be performed by perfluorooctanol treatment, surfactant dilution with surfactant-free continuous phase or electrostatic pulses. means selected from, preferably by using an electrostatic-pulses gun (Zerostat 3, Milty, UK).

In another embodiment of the digital multiplex method of the invention, when the particles are separated from each other in a solution, for example in an aqueous solution, there is no need to perform the recovering step f1) and the detection and/or the quantification in step g) may be performed directly.

In order to detect and/or measure the signal of the particles including barcode signal and the amplification signal of each particle in step g) of the digital multiplex method of the present invention, it is necessary to barcode (or to label) said particles. As indicated above, the barcoding is performed in step b) of the digital multiplex method of the present invention or concomitantly with the functionalization of the particles in step a). Many different barcodes may be used for this purpose. As indicated above, a number of methods have been developed in the art to barcode particles. Such methods are disclosed in references [28]-[32]. For example, such methods may be selected from fluorescent dyes which may be grafted (covalently or not), or directly integrated to the particle by mixing it to the monomer mixture, as it can be done with quantum dots; flow lithography, photonic crystals and rare-earth ions integrations. The skilled artisan would be able to adapt the conventional barcodes means to the digital method of the present invention.

According to one preferred embodiment of the digital multiplex method of the invention, the used barcode is a fluorescent signal. During the incubation for amplifying the signal sequence in step f), particles-binding targeted biomolecule trigger the amplification reaction that in turn induces the activation of the reporting template, preferably of the fluorescence probe. Thus, according to one embodiment of the digital multiplex method of the present invention, step g) of detecting and/or measuring the barcode signal comprises detecting and/or measuring the barcode signal for each particle, associated to the target biomolecule, and simultaneously the signal of the reporting template related to the amplification signal, wherein said signal is preferably a fluorescence signal.

In one embodiment, when the digital multiplex method of the invention is used for measuring the absolute concentration of the target biomolecules in the tested biological sample, the fluorescent particle capturing the target biomolecule and the non-fluorescent particles are counted and their ratio is calculated.

Preferably, the analysis by flow cytometry permits the counting of positive (presence of the target)/negative (absence of the target) particles, simultaneously with the measurement of the barcode signals, and therefore the calculation of the exact concentrations of the multiple target biomolecules in the initial sample.

The positive population corresponds to particles having captured at least one target biomolecules. Considering a Poissonian random capture of the targets by each particle population, it is possible to compute the concentration of the associated target biomolecule by first calculating the parameter λ of the Poissonian distribution:

$${\sum }_{k = 1}^{∞} \frac{{λ}^{k}}{k !} {e}^{- λ} = {F}_{pos}$$

where λ is the average number of target biomolecules per particle, k is the number of target biomolecules captured by one particle and Fₚₒₛ is the fraction of positive particles (particles that captured at least one target biomolecule). From this equation the inventors deduced:

λ=−Ln (1−Fₚₒₛ)

Then, the measured concentration of target biomolecule may be given by the following equation:

[Let7a]=[part]·λ[Let7a]=[part]·ln(1−Fₚₒₛ)

where [part] is the initial concentration of particles.

For example, the counting and the analysis in the digital multiplex method of the invention may be performed by flow cytometry in an Attune N×T (Thermo Fisher Scientific, MA, USA).

According to another embodiment, another conventional method, for example fluorescence microscopy may be used for counting of positive/negative particles.

According to preferred embodiment, the digital multiplex method of the present invention comprises the steps of:

a) functionalizing a suspension of particles, preferably of microparticles with a first oligonucleotide which is a conversion oligonucleotide (cT) and with a second oligonucleotide which is a reporting oligonucleotide (rT);

b) adding to the particles functionalized in step a) barcodes allowing the discrimination of the particles targeting multiple biomolecules;

c) contacting the particles obtained in step b) with the tested sample to capture the multiple target biomolecules;

d) resuspending the particles having captured or not the target biomolecules in an amplification mixture including a buffer, enzymes, a third oligonucleotide which is an amplification oligonucleotide (aT), a fourth oligonucleotide which is a leak absorption oligonucleotide (pT) and deoxy-nucleoside triphosphate (dNTPs);

e) separating the particles in the suspension obtained in step d) from each other so that each particle can react independently;

f) incubating the isolated particles at a constant temperature so that each target biomolecule triggers an amplification reaction and a barcode signal;

f1) recovering the particles, and

g) detecting and/or measuring the signals of the particles including the barcode signal and the amplification signal of each particle.

A schema showing the principle of this preferred embodiment of the digital multiplex method of the invention is set forth in FIG. 1.

The specific technical features of the digital multiplex method according to this preferred embodiment are those detailed above.

### Use of the Method of Digital Multiplex Detection and/or Quantification of the Invention for Diagnosis Purpose

The biomolecules mentioned above may be present in all type of sample. For example, they may be present in a sample obtained from non-living organism (for example soil sample, water sample, air sample, food sample etc.) or in samples obtained from living organism, for example, cells, body fluids or tissues. The biomolecules mentioned above may be present for example in all bodily fluids and thus they are accessible via minimally invasive liquid biopsies (serum, plasma, urine).

According to one embodiment of the present invention, the targeted biomolecules detected and/or measured by the digital multiplex method of the invention are used as biomarkers.

In the context of the present invention, the term “biomarker” relates to a naturally occurring molecule, preferably a biomolecule, protein, enzyme, gene, nucleic acid or characteristic by which a particular pathological or physiological process, disease, etc. can be identified.

These biomarkers may be used for detecting diseases in living organisms such as plants, animals, preferably a mammal and more preferably a human.

Moreover, these biomarkers may be used for detecting one or several anomalies and food and agri-food industry or in the environment.

The biomarkers may be present for example in all bodily fluids and thus accessible via minimally invasive liquid biopsies (serum, plasma, urine, tears, saliva, sweat, etc.).

Preferably, they are used as biomarkers for detecting diseases selected from the group comprising cancer, neuronal diseases, cardiovascular diseases, inflammatory diseases, autoimmune diseases, diseases due to viral or bacterial infection skin diseases, skeletal muscle diseases, dental diseases and prenatal diseases.

In the context of the present invention, the term “detection” is used for defining in general manner the detection of target biomolecule in a sample as defined above.

As used herein, the term “detection” also relates to the diagnosis or the prognosis of one or several of the above cited diseases or of their symptoms. The detection also includes the prediction of one or several of said diseases or of the prediction of the risk for a subject to develop one or several of these diseases.

Moreover, the term “detection” further relates to the agro-diagnosis, i.e to the diagnosis of phytopathologies, particularly of phytopathologies having biotic of abiotic origin as defined in the present invention.

In the context of the present invention, the term “cancer” refers to a malignant neoplasm characterized by deregulated or uncontrolled cell growth. In particular, a “cancer cell” refers to a cell with deregulated or uncontrolled cell growth.

The term “cancer” includes primary malignant tumours (e. g., those whose cells have not migrated to sites in the subject's body other than the site of the original tumor) and secondary malignant tumors (e. g., those arising from metastasis, the migration of tumour cells to secondary sites that are different from the site of the original tumour). Such cancer may notably be selected from the group of solid cancers and/or from the group of hematopoietic cancers.

In one embodiment of the invention, the cancer is selected from osteolysis, bone sarcomas (osteosarcoma, Ewing's sarcoma, Giant cell tumours of bone), bone metastases, glioblastoma and brain cancers, lung cancer, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphangioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukaemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In the context of the invention, the term “neuronal diseases” refers to the diseases of the central and peripheral nervous system comprising the brain, spinal cord, cranial nerves, peripheral nerves, nerve roots, autonomic nervous system, neuromuscular junction, and muscles. The neuronal diseases are selected from neurodevelopmental, neurodegenerative or psychiatric diseases. They include epilepsy, Alzheimer disease and other dementias, cerebrovascular diseases including stroke, migraine and other headache disorders, multiple sclerosis, Parkinson's disease, neuroinfections, brain tumours, traumatic disorders of the nervous system due to head trauma etc.

As used herewith “cardiovascular diseases” relate to heart or vessel failure including coronary heart disease: disease of the blood vessels supplying the heart muscle; cerebrovascular disease: disease of the blood vessels supplying the brain; peripheral arterial disease: disease of blood vessels supplying the arms and legs; rheumatic heart disease: damage to the heart muscle and heart valves from rheumatic fever, caused by streptococcal bacteria; congenital heart disease: malformations of heart structure existing at birth and deep vein thrombosis and pulmonary embolism: blood clots in the leg veins, which can dislodge and move to the heart and lungs.

As used herein, “inflammatory disease” preferably refers to acute pancreatitis; ALS; Alzheimer's disease; cachexia/anorexia; asthma; atherosclerosis; chronic fatigue syndrome, fever; diabetes (e.g., insulin diabetes); glomerulonephritis; graft versus host rejection; hemorrhagic shock; hyperalgesia, inflammatory bowel diseases; inflammatory conditions of a joint, including osteoarthritis, psoriatic arthritis and rheumatoid arthritis; ischemic injury, including cerebral ischemia (e.g., brain injury as a result of trauma, epilepsy, hemorrhage or stroke, each of which may lead to neurodegeneration); lung diseases (e.g., ARDS); multiple myeloma; multiple sclerosis; myelogenous (e.g., AML and CML) and other leukemias; myopathies (e.g., muscle protein metabolism, esp. in sepsis); osteoporosis; Parkinson's disease; pain; pre-term labor; psoriasis; reperfusion injury; septic shock; side effects from radiation therapy, temporal mandibular joint disease, tumor metastasis; or an inflammatory condition resulting from strain, sprain, cartilage damage, trauma, orthopedic surgery, infection or other disease processes.

In the context of the present invention “autoimmune diseases” are defined as conditions wherein the body of a subject produces antibodies directed against his own tissues and cells. Examples of autoimmune disease are: type I diabetes, Graves' disease, inflammatory bowel disease, multiple sclerosis, psoriasis, rheumatoid arthritis, systemic lupus erythematosus etc.

The diseases du to viral or bacterial infection are caused by pathogenic virus or bacterial strains. Example of such diseases are AIDS, ascariasis; athletes' foot; bacillary dysentery, chickenpox; cholera, common cold, dengue, diarrhea, diphtheria, filariasis, gonorrhea, herpes, hook worm disease, influenza flu, leprosy, measles, mumps, oriental sore, pinworm disease, plague, pneumonia, poliomyelitis, rabies, ringworm, septic sore throat, sleeping sickness, smallpox, syphilis, tetanus, typhoid, vaginitis, viral encephalitis, whooping cough, etc.

The “skin diseases” are conditions affecting the skin as for example acne, alopecia areata, basal cell carcinoma, bowen's disease, congenital erythropoietic porphyria, contact dermatitis, darier's disease disseminated superficial actinic porokeratosis, dystrophic epidermolysis bullosa, eczema (atopic eczema), extra-mammary paget's disease, epidermolysis bullosa simplex, erythropoietic protoporphyria, fungal infections of nails, hailey-hailey disease, herpes simplex, hidradenitis suppurativa, hirsutism, hyperhidrosis, ichthyosis, impetigo, keloids, keratosis pilaris, lichen planus, lichen sclerosus, melanoma, melasma, mucous membrane pemphigoid, pemphigoid, pemphigus vulgaris, pityriasis lichenoides, pityriasis rubra pilaris, plantar warts (verrucas), polymorphic light eruption, psoriasis pyoderma gangrenosum, rosacea, scabies, shingles, squamous cell carcinoma, sweet's syndrome, urticaria and angioedema, vitiligo

As used herein, the “skeletal muscular diseases” relates to diseases of bones, muscles (myopathy) and skeletal-muscular junction. For example, such diseases are selected from back pain, bursitis, fibromyalgia, fibrous dysplasia, Injuries to the growth plate, heritable disorders of connective tissue, Marfan syndrome, osteogenesis imperfecta, osteonecrosis, osteoporosis, Paget's disease of bones, scoliosis, spinal stenosis, tendinitis etc.

As used herein the “dental diseases” relate to dental and mouth issues. Example of such diseases are dental cavities, periodontal (gum) disease, oral cancer, oral infectious diseases, trauma from injuries, and hereditary lesions, etc.

As used herein the “prenatal diseases” relate to diseases that could impact pregnancy or fetal development such as AIDS, amniotic fluid, bleeding during pregnancy, cervix disorders, pregnancy diabetes, disseminated intravascular coagulation (DIC), ectopic pregnancy, erythroblastosis fetalis, fetal development issues, high blood pressure in pregnancy, HELLP syndrome, hydatidiform mole, hyperemesis gravidarum, intrauterine growth restriction, large for gestational age (LGA), miscarriage, placenta abruptio, Placenta previa, placental insufficiency, polyhydramnios, prenatal testing, pregnancy loss, preterm labor and birth, rubella, small for gestational age (SGA), systemic lupus erythematosus, toxoplasmosis, twin-to-twin transfusion syndrome, twins, triplets, multiple births, vaginal bleeding during pregnancy etc.

The digital multiplex method of the present invention may be thus used in the in vitro diagnosis methods for diagnosing diseases selected from the group comprising cancer, neuronal diseases, cardiovascular diseases, inflammatory diseases, autoimmune diseases, diseases due to viral or bacterial infection skin diseases, skeletal muscle diseases, dental diseases and prenatal diseases.

In the second aspect, the present invention thus relates to an in vitro method for diagnosis of a disease selected from the group comprising cancer, neuronal diseases, cardiovascular diseases, inflammatory diseases, autoimmune diseases, diseases due to viral or bacterial infection skin diseases, skeletal muscle diseases, dental diseases and prenatal diseases. comprising the use of the digital multiplex method according to the invention.

According to one embodiment, said diagnosis method comprise the steps of:
providing a sample obtained from a subject, and
detecting the presence or the absence of one or more of said diseases by the use of the digital multiplex method of the invention.

The specificity, the sensitivity, the simplicity and the rapidity of the digital multiplex method of the invention allow to use it in agro diagnosis methods, particularly for diagnosis of diseases caused by biotic stress such as infectious and parasitic diseases, or caused by abiotic stress such as nutritional deficiencies or unfavorable environments.

According to the third aspect, the present invention also relates to an in vitro method for agro diagnosis of a disease selected from the group comprising:
diseases caused by biotic stress, preferably by infectious and/or parasitic origin, or
diseases caused by abiotic stress, preferably caused by nutritional deficiencies and/or unfavorable environment,

said method comprising the use of the multiplex digital method of the invention.

According to one embodiment, said diagnosis method comprise the steps of:
providing a sample obtained from any one of plant's parts, and
detecting the presence or the absence of one or more of said diseases by the use of the digital multiplex method of the invention.

In the context of the present invention, the term “agro diagnosis” relates to a diagnosis of phytopathologies, said diagnosis comprising carrying out an analysis from plant samples for an identification of fungi, viruses, bacteria, nematodes and any other life organism causing biotic stress and/or for identifying biomolecules which presence is due to an abiotic stress.

In the context of the present invention, the term “phytopathology” or “plant disease” relates to plant anomalies which are manifested by changes in plant morphology, physiology or behavior due to a biotic or to an abiotic stress.

As used herein, the term “biotic stress” relates to stress or also to diseases caused by life organisms. The biotic stress may be caused by any living organism but has preferably an infectious and/or parasitic origin and may be caused by organisms selected from fungi, viruses, bacteria and nematodes.

According to one embodiment, the agro diagnosis method of the invention may be used for diagnosis of diseases caused by fungi, said diseases being selected from anthracnose, black knot, blight, chestnut blight (such as late blight), canker, clubroot, damping-off, Dutch elm disease, ergot, Fusarium wilt, Panama disease, leaf blister, mildew (such as downy mildew and powdery mildew), oak wilt, rot (such as basal rot, gray mold rot and heart rot), rust (such as blister rust, cedar-apple rust and coffee rust), scab (such as apple scab), smut, bunt, corn smut, snow mold, sooty mold and Verticillium wilt.

According to another embodiment, the agro diagnosis method of the invention may be used for diagnosis of diseases caused by viruses, said diseases being selected from curly top, mosaic, psorosis and spotted wilt.

According to still another embodiment, the agro diagnosis method of the invention may be used for diagnosis of diseases caused by bacteria, said diseases being selected from aster yellows, bacterial wilt, blight (such as fire blight and rice bacterial blight), canker, crown gall, rot, basal rot and scab.

The agro diagnosis method of the invention may also be used for diagnosis of diseases caused by nematodes selected from root-knot nematodes (such as Meloidogyne spp.), cyst nematodes (such as Heterodera and Globodera spp.), root lesion nematodes (such as Pratylenchus spp.), the burrowing nematode (such as Radopholus similis), Ditylenchus dipsaci, the pine wilt nematode (such as Bursaphelenchus xylophilus); the reniform nematode (such as Rotylenchulus reniformis), Xiphinema index, Nacobbus aberrans and Aphelenchoides besseyi.

According to one embodiment, the agro diagnosis method of the present invention is used for diagnosing diseases caused by “abiotic stress” which term defines the diseases which are not caused by life organisms. These diseases are preferably caused by nutritional deficiencies and/or unfavorable environment. For example, the abiotic stress may be caused by inappropriate pH, water availability (drought stress), temperature (heat stress and cold stress), oxygen and/or gases availability, mineral deficiencies (salinity stress) and toxics compounds (for example pollutants).

The specificity, the sensitivity, the simplicity and the rapidity of the digital method of the invention allow to also use it in methods for detection of biomolecules in the field of food, agri-food industry and in the environment. Particularly, the digital method of the present invention is used for detecting food, agri-food and environmental anomaly. This detection is performed by using the digital method of the invention for detecting biomolecules which may be considered as biomarkers for said anomalies.

Such biomolecules may be for example a part of living organism or may be produced by the activity of a living organism or also may be artificial biomolecules. These biomolecules (or biomarkers) are selected from the group comprising biopolymers, particularly, DNA, RNA, proteins and enzymes.

Said biomolecules are present in agri-foods and in foods in an original and/or in a transformed product.

They also may be present in the environment, for example, in the air, in the water and/or in the soil.

Thus, according to one aspect, the present invention also relates to in vitro methods for detecting biomolecules (biomarkers) in agri-food, in food industry and/or in environment, said methods comprising the use of the digital method of the invention.

In the context of the present invention, the term “food” relates to all food, basic or transformed, produced without using industrial process or by using such process.

In the context of the present invention, the term “agri-food”, relates to the agri-food industry, i.e. to the commercial production of food by farming.

The terms “environment” or “environmental” relate to natural environment, i.e an ecological units that function as natural systems without massive civilized human intervention, including all vegetation, microorganisms, soil, rocks, atmosphere, and natural phenomena that occur within their boundaries and their nature. These terms also relate to no natural or artificial environment which may be created by the human. According to one aspect, the present invention also relates to an in vitro method for detecting anomalies in a food and in an agri-food industry and/or in the environmental using the digital method of the invention.

Preferably, said method comprises:
providing a sample obtained from a food, from an agri-food or from the environment,
detecting a tested biomolecule (biomarker) in said sample by the digital method of the invention.

Kit for Implementing the Digital Multiplex Method of the Present Invention

The present invention also relates to a kit which may be used for detecting and/or quantifying multiple target biomolecules accordingly to the method of the invention.

According to the fourth aspect, the present invention relates to a kit for detecting and/or quantifying multiple target biomolecules comprising:

a) a suspension of particles, preferably of microparticles functionalized with a one or more oligonucleotides selected from a first oligonucleotide which is a conversion oligonucleotide (cT), a second oligonucleotide which is a reporting oligonucleotide (rT), a third oligonucleotide which is an amplification oligonucleotide (aT) and a forth oligonucleotide which is a leak absorption oligonucleotide (pT), to which particles are added different barcodes allowing the discrimination of the particles targeting different biomolecules;

b) a mixture of enzymes, preferably selected from the group comprising polymerase, nicking enzyme or restriction enzyme and exonuclease and optionally oligonucleotides, and c) a separating agent.

In particular embodiment, the present invention relates to a kit comprising:

a) a suspension of particles, preferably of microparticles functionalized with a first oligonucleotide which is a conversion oligonucleotide (cT) and with a second oligonucleotide which is a reporting oligonucleotide (rT) to which particles are added barcodes allowing the discrimination of the particles targeting different biomolecules;

b) a mixture of enzymes, preferably selected from the group comprising polymerase, nicking enzyme or restriction enzyme and exonuclease and of oligonucleotides comprising a third oligonucleotide which is an amplification oligonucleotide (aT) and a fourth oligonucleotide which is a leak absorption oligonucleotide (pT), and

c) separating agent, preferably a water-in-oil emulsion.

According to still another embodiment, the components in point b) above are provided separately, i.e., the oligonucleotides are provided separately from the enzymes and are mixed later.

The constituent elements described in points a), b) and c) of the kit correspond to the same used in the digital multiplex method of the invention as described above.

The present invention also relates to the use of said kit for implementing the digital multiplex method of the present invention.

The kit of the present invention may also comprise instructions for use.

The particular embodiments of the present invention will appear clearly from the examples and the figures below.

### FIGURES

FIG. 1. Principle of multiplex and digital detection of microRNA. A collection of barcoded particles are functionalized with target-specific conversion oligonucleotide and a reporting oligonucleotide. The particles are mixed with the sample, leading to the random capture of the target biomolecules by the particles. After washing, the particles together with the amplification and leak absorption oligonucleotides and the enzymatic mix. are compartmentalized (e.g. in water-in-oil droplets). After incubation at constant temperature (e.g. 50° C.), the particles are analyzed by flow cytometry, giving access to absolute concentration of each target, computed from the proportion of positive/negative particles for each population.

FIG. 2. Results of the flow cytometry analysis for the detection of Let7a. a. Particle B_{Let7a} before incubation. b. Particle B_{Let7a} after incubation at 50° C. for 4 hours (NC=no target). c. Particle B_{Let7a} after incubation at 50° C. for 4 hours in presence of 1 pM of target. d. Measured concentrations computed from the ratio of positive particles Fₚₒₛ:[Let7a]=[part] ln(1−Fₚₒₛ).

FIG. 3. Microscopy vs flow cytometry readout. a. Fluorescence images of the encapsulated particles after incubation. The white dots correspond to the particles (positive and negative) that bear a FAM-modified rT and the positive droplets appear in gray, corresponding to the fluorescence of Atto633-modified probe in solution. The microRNA was detected in the gray droplets and not in the dark gray ones. b. Histograms from the flow cytometry readout. The microRNA was detected in 79% of bead-containing droplets. c. Comparative analysis of microscopy (statistics computed from 250 bead-containing droplets) and flow cytometry readout.

FIG. 4. Detected concentrations depending on the particles' concentration in the encapsulated mix.

FIG. 5. Enzyme trapping by on-particles oligonucleotides. An enzyme mixture (polymerase, nickases, exonuclease) is incubated with or without particles grafted or not with oligonucleotides. After 30 minutes of incubation at 30° C., the particles are pelleted and the supernatant is mixed with an in-solution molecular program (amplification and pseudo template oligonucleotides) and spiked with 1 pM of Let7a. The samples are incubated at 50° C. and the fluorescence of the rT monitored in real-time. a. Real-time fluorescence curves showing the amplification reaction. b. Extraction starting times from the fluorescence curves.

FIG. 6. Effect of the neutral particles on the false positive and true positive rate. a. Cytometry fluorescence histograms of the negative controls (without target, top row) and of the positive controls (with 1 pM Let7a, bottom row) depending on particles' concentration. b. Percentages of positive particles as function on the overall particles' concentration; c. target biomolecules concentration in positive particles.

FIG. 7. Let7a range detection using neutral particles. 10⁵ B_{Let7a} supplemented with 2·10⁵ B_{N} were used for the quantification of 0, 0.2 and 1 pM of Let7a a. Cytometry fluorescence histograms. b. Comparison of measured Let7a concentrations depending on theoretical spiked-in concentration.

FIG. 8. Detected Let7a and miR92a concentrations in a 2-target assay. a. Cytometry fluorescence histograms of the 3 particles populations (B_{Let7a}, B₉₂ₐ, B_{N}) according to their fluorescent barcode (Atto633). b. Cytometry fluorescence results (rT fluorescence) of B92a and B_{Let7a}. c. Measured concentration of the 2 targets.

FIG. 9. Triplex assay for the simultaneous quantification of Let7a, mir92a and mir203a. a. Fluorescence intensity of the barcode (Atto633) for each population of particles (flow cytometry measurement). Particles populations are, from left to right: b. Neutral particles, miR92a particles, miR203a particles and Let7a particles and cytometry fluorescence signals of miR92a, miR203a and Let7a particles depending on the sample c. Measured concentrations of each microRNA target.

FIG. 10. Flow cytometry discrimination of ten particles populations using two-dimensional fluorescent barcoding. 1 μm streptavidin-coated particles are functionalized with various ratio of biot-TTTT-FAM (5 levels) and biot-DyXL510 (2 levels)

FIG. 11. Let7a detection from human colon total RNA extract. a. Cytometry fluorescence signals of Let7a particles. b. Measured concentrations of Let7a in the negative control and the sample containing 10 ng/μL of colon total RNA.

FIG. 12. Dynamic range adjustment as a function of the number of particles. This graph shows the evolution of the dynamic range for sample of 20 μL of various target concentration. The dynamic range is comprised between the limit of detection (LoD) and the higher limit of quantification (hLoQ). For the sake of simplicity, it is assumed that a LoD is equal to the limit of blank (LoB=average percentage of false positive events) arbitrarily set a 5%. The hLoQ is arbitrarily set at 95% of positive events (meaning that above this value, the quantification is considered not reliable). The bars at the bottom represent the appropriate number of particles to be used for each target concentration to fall within the dynamic range. As a result, it is possible to tune the dynamic range.

FIG. 13. Detected concentration of Let7a depending on the presence/absence of Klenow DNA polymerase (3′45′ exo-) during the capture on the particles.

FIG. 14. Background amplification reduction effect of strengthening “hard” washing procedure.

FIG. 15. Designing of poly(T) conversion template. The expected concentrations are 0 M (Negative controls) and 1.00 E-12 M (1 pM sample).

FIG. 16. Comparison of expected and measured patterns on a 6-plex miRNA detection.

FIG. 17. Tunable dynamic range.

FIG. 18. Detection of 3 microRNAs from human total RNA.

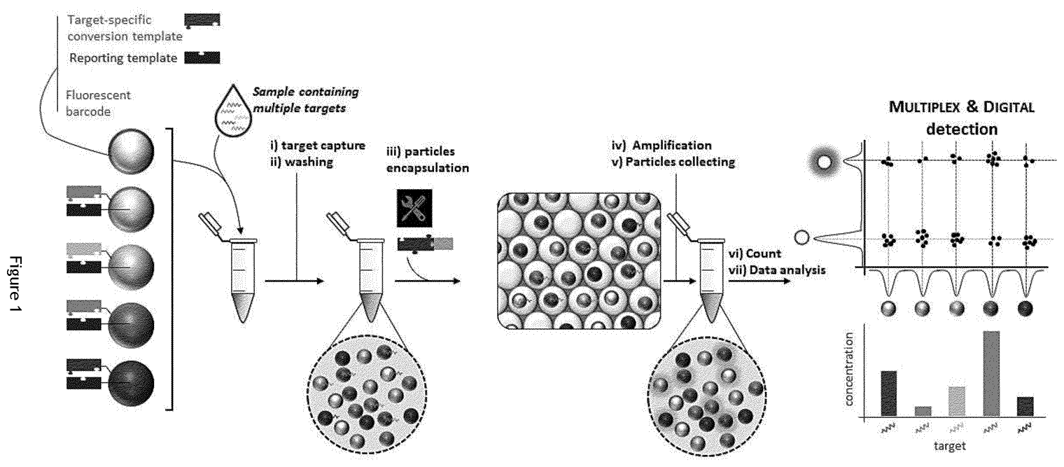

### EXAMPLES

Methods and Materials

Oligonucleotides

All oligonucleotides (templates and synthetic micro RNA) used in the present invention were purchased from Biomers (Germany). The oligonucleotides sequences were purified by HPLC.

Template sequences are protected from degradation by the exonuclease, by 5′ phosphorothioate modification. cT (conversion template) and rT (reporting template) are modified by a polythymidylate linker followed by a biotin moiety in 3′ and 5′ respectively.

Said oligonucleotides are shown in Table 2 below:

TABLE 2
Oligonucleotide sequences used in the invention. “*” denotes phosphorothioate
backbone modification. “p” denotes a 3′ phosphate modification. “biotin” and “bioteg”
refer to biotinylated synthons, respectively using aminoethoxy-ethoxyethanol linker and the
longer triethylene glycol linker. Upper and lower cases represent deoxyribonucleotide
and ribonucleotide, respectively. “aT” corresponds to autocatalytic template; “pT” corresponds
to pseudo template, “rT” corresponds to reporting template and “cT” corresponds to
conversion template. Atto633, FAM, DyXL510 are fluorophores. dTFAM is a deoxythymidine
nucleoside derivitized with 6-FAM (6-carboxyfluorescein) through a spacer arm.

| SEQ | | | |
|---|---|---|---|
| ID | | | |
| NO: | Name: | Sequence | Function |
| 60 | bc | CATTCTGGACTG | signal |
| 61 | aTc | C*A*G*T*CCAGAATGCAGTCCAGAA p | aT |
| 62 | pTbc | T*T*T*T*TCAGTCCAGAATG p | pT |
| 63 | rTbc | Atto633 *A*T*TCTGAATGCAGTCCAGAAT BHQ2 | rT |
| 64 | rTbc-biot | Biotin *T*T*TTTTTTT dTFAM | rT |
| | | GTGAGAATGCAGTCCAGAATGTCTCAC BHQ2 | |
| 65 | cTbc-Let7a-biot | TGCAGTCCAGAAGTTTGACTCAAACTATACAACCTACTACCT | cT |
| | | CATTTTTTT biotin | |
| 66 | cTbc-92a-biot | TGCAGTCCAGAAGTTTGACTCAAGCATTGCAACCGATCCCAA | cT |
| | | CCTTTTTTT biotin | |
| 67 | cTbc-203a-biot | TGCAGTCCAGAAGTTTGACTCAACTAGTGGTCCTAAACATTT | cT |
| | | CACTTTTTTT biotin | |
| 68 | cTbc-Let7a | TGCAGTCCAGAAGTTTGACTCAAACTATACAACCTACTACCT | cT |
| | | CA p | |
| 69 | Let7a | ugagguaguagguuguauaguu | microRNA |
| 70 | mir92a | agguugggaucgguugcaaugcu | microRNA |
| 71 | mir203a-3p | gugaaauguuuaggaccacuag | microRNA |
| 72 | Let7a-D | TGAGGTAGTAGGTTGTATAGTT | DNA analogue of microRNA |
| 73 | mir92a-D | AGGTTGGGATCGGTTGCAATGCT | DNA analogue of microRNA |
| 74 | mir203a-D | GTGAAATGTTTAGGACCACTAG | DNA analogue of microRNA |
| | mir10a | uacccuguagauccgaauuugug | microRNA |
| | mir16 | uagcagcacguaaauauuggcg | microRNA |
| | mir21 | uagcuuaucagacugauguuga | microRNA |
| | lin4 | ucccugagaccucaaguguga | microRNA |
| | T5-biot-Atto633 | Atto633 TTTTT bioteg | barcode |
| | T5-biotFAM | FAM TTTTT bioteg | barcode |
| | T5-biot-DyXL510 | DyXL510 TTTTT bioteg | barcode |

Particle Functionalization

Streptavidin-coated 1 μm particles (Dynabeads C1) were obtained from Invitrogen. Prior to functionalization, particles were washed three times in a washing buffer (20 mM Tris-HCl pH 7.5, 1 M NaCl, 1 mM EDTA, 0.2% Tween20 (Sigma-Aldrich)) and then resuspended in the same buffer. The biotinylated oligonucleotides are added to the particle suspension, mixed thoroughly with a vortex and incubated for 15 minutes at room temperature. After functionalization, the particles were washed once in the washing buffer, once in the storage buffer (5 mM Tris-HCl pH 7.5, 50 mM NaCl, 500 μM EDTA, 5 mM MgSO₄) and finally resuspended in the storage buffer. Until use, grafted particles are stored at 4° C.

microRNA Capture

All capture mixes were assembled at 4° C. in 200 μL PCR tubes. The particles are mixed in the reaction buffer (10⁹ beads/mL for each bead population, 20 mM Tris HCl pH 8.9, 10 mM (NH₄)₂SO₄, 40 mM KCl, 10 mM NaCl, 10 mM MgSO₄, 25 μM each dNTP, 0.1% (w/v) Synperonic F 104, 2 μM Netropsin). The samples were spiked with synthetic microRNA targets (serially diluted in 1× Tris-EDTA buffer using Low DNA retention tips) or target-containing fluids (plasma, urine, cell extracts, tissue extract . . . ). The samples were incubated for 1 hour at 30° C. under 2000 rpm stirring in a ThermoMixer (Eppendorf). The particles were then pelleted and resuspended in the storage buffer at a concentration of 10⁹ particles/mL. Alternatively, the capture step can be skipped by injecting the particles and the targets directly in the detection mix (see below).

Reaction Mixture Assembly

All reaction mixtures were assembled at 4° C. in 200 μL PCR tubes. The templates (aT and pT) and the particles (3·10⁸ part/mL including the neutral and detection particles) were mixed with the reaction buffer (20 mM Tris HCl pH 8.9; 10 mM (NH₄)2SO₄, 40 mM KCl, 10 mM NaCl, 10 mM MgSO₄, 25 μM each dNTP, 0.1% (w/v) Synperonic F 104, 2 μM Netropsin) and the BSA (200 μg/mL) together with the enzymes (200 u/mL Nb.Bsml, 10 u/mL Nt.BstNBI, 80 u/mL Vent(exo-) and 23 nM ttRecJ).

Droplets Generation and Incubation

A 2-inlet (one for the oil, one for the aqueous sample) flow-focusing microfluidic mold was prepared with standard soft lithographic techniques using SU8 photoresist (MicroChem Corp., MA, USA) patterned on a 4-inch silicon wafer. A 10:1 mixture of Sylgard 184 PDMS resin (40 g)/crosslinker (4 g) (Dow Corning, MI, USA) was poured on the mold, degassed under vacuum and baked for 2 hours at 70° C. After curing, the PDMS was peeled off from the wafer and the inlets and outlet holes of 1.5 mm diameter were punched with a biopsy punch (Integra Miltex, PA, USA). The PDMS layer was bound onto a 1 mm thick glass slide (Paul Marienfelf GmbH Et Co. K.G., Germany) immediately after oxygen plasma treatment. Finally, the chip underwent a second baking at 200° C. for 5 hours to make the channels hydrophobic. The aqueous sample phase (amplification mix+particles) and the continuous phase (fluorinated oil Novec-7500, 3 M containing 1% (w/w) fluorosurfactant (Emulseo, France)) were mixed on chip using a pressure controller MFCS-EZ (Fluigent, France) and 200 μm diameter tubing (C.I.L., France) to generate 0.5 μL droplets by hydrodynamic flow focusing. The droplets were transferred to PCR tubes and incubated at 50° C. to let the amplification reaction happen.

Particle Analysis

After incubation, the droplets were mixed with surfactant-free fluorinated oil (Novec 7500) (1:5 v/v). The emulsion was broken using an electrostatic-pulses gun (Zerostat 3, Milty, UK). Once all water droplets merged in one water drop, the oil phase was discarded and the water drop was resuspended in the sheath fluid (Attune N×T Focusing Fluid, Thermo Fisher Scientific, MA, USA). The sample was analyzed by flow cytometry in an Attune N×T (Thermo Fisher Scientific, MA, USA).

### Example 1: DNA-Grafted Particle for the Digital Detection of Micro RNA

The digital multiplex strategy of the present invention relies on the use of DNA-grafted particles able to capture single nucleic acid targets, trigger an exponential amplification reaction and report a positive signal. The ratio of positive versus negative particles gives access to the absolute concentration of the target in the initial sample, computed from the Poisson law. The inventors firstly investigated the possibility to detect one microRNA target using this digital approach.

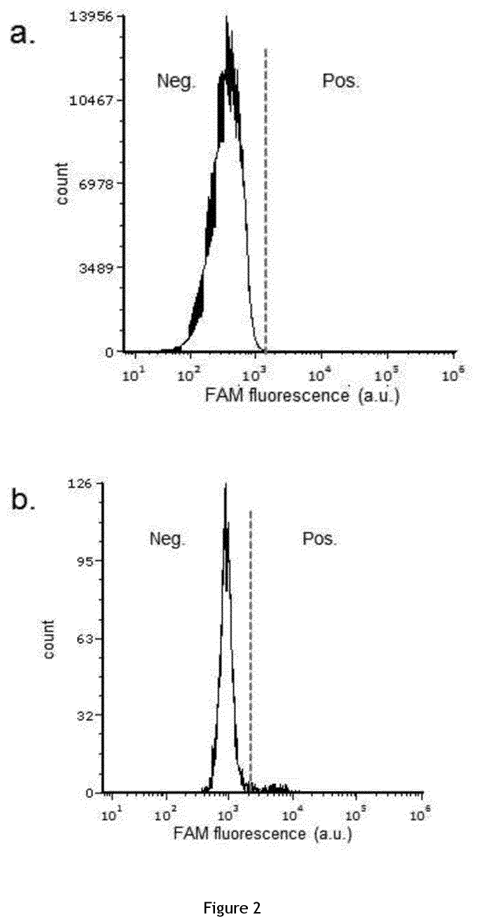

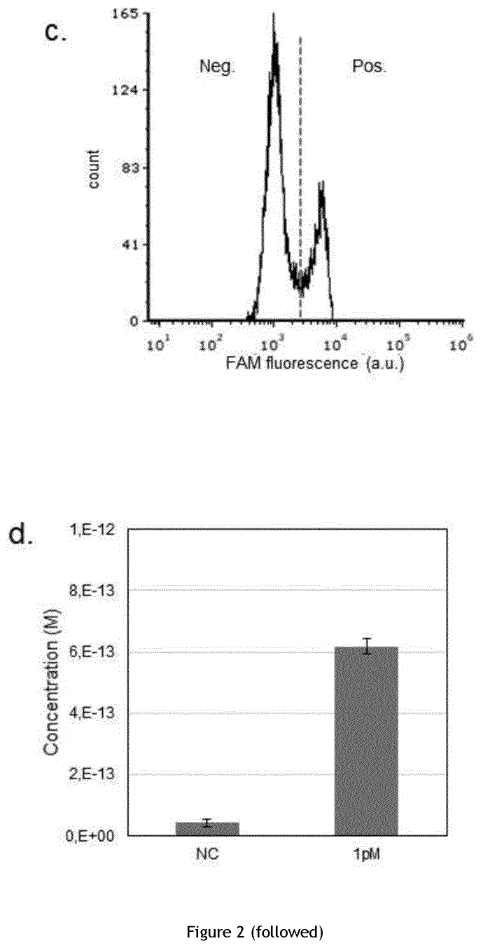

For that, 1 μm streptavidin-coated magnetic particles were functionalized with the conversion template (cT, SEQ ID 65) targeting the microRNA Let7a and the reporting template (rT, SEQ ID 64). The particles are mixed with a sample containing 0 or 1 pM of the synthetic Let7a RNA sequence (SEQ ID 69), together with the amplification machinery (aT, SEQ ID 61 pT, SEQ ID 62, enzymes, buffer and dNTP). The suspension is then encapsulated using a microfluidic flow focusing junction, allowing for the compartmentalization of individual particles in water-in-oil droplets. Upon incubation at 50° C., the particle having captured at least one microRNA start producing multiple copies of a short DNA sequence (called the signal) via polymerization/nicking cycle, which eventually trigger the amplification reaction. In turn, the output strand of this reaction hybridizes to supported probe, which leads to the fluorescence increase of the particle. The particles are finally recovered by breaking the emulsion and analyzed by flow cytometry, whose results are presented in FIG. 2. It may be noted that without incubation (and thus, without amplification FIG. 2a), a single population of weakly fluorescent particles is observed, corresponding to the background amplification of the supported probes. After incubation in presence of 1 pM of Let7a, a population of highly fluorescent particle (31%) is distinguished from the negative population (69%). This positive population corresponds to particles having captured at least one target. Assuming a Poissonian random capture of the targets by the particle, it is possible to compute the concentration of Let7a measured with the assay, by first calculating the parameter λ of the Poissonian distribution:

$${\sum }_{k = 1}^{∞} \frac{{λ}^{k}}{k !} {e}^{- λ} = {F}_{pos}$$

where λ is the average number of targets per particle, k is the number of targets captured by one particle and Fₚₒₛ is the fraction of positive particles (particles that captured at least one target). Then, the measured concentration of Let7a ([Let7a]) is given by the following equation:

[Let7a]=[part]·λ[Let7a]=[part]·ln (1−F)

where [part] is the initial concentration of particle. From this equation the inventors deduced:

λ=−Ln (1−Fₚₒₛ)

Hence, the measured concentration in the initial sample is 620 fM, which is in accordance with the theoretical concentration (1 pM) given the incertitude on the target dilution (diluted from 100 μM stock solution). The negative control reports 2.6% of false positive particles.

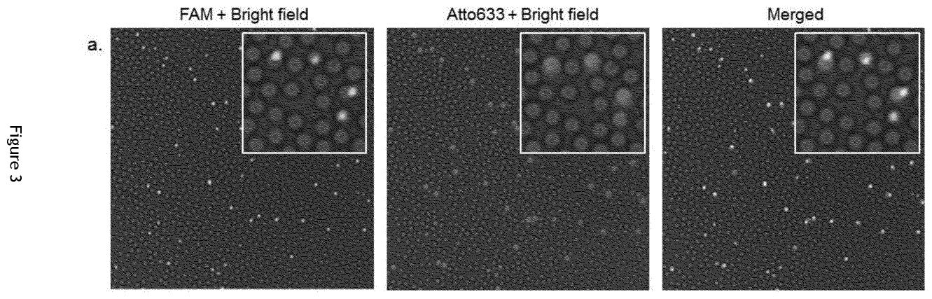

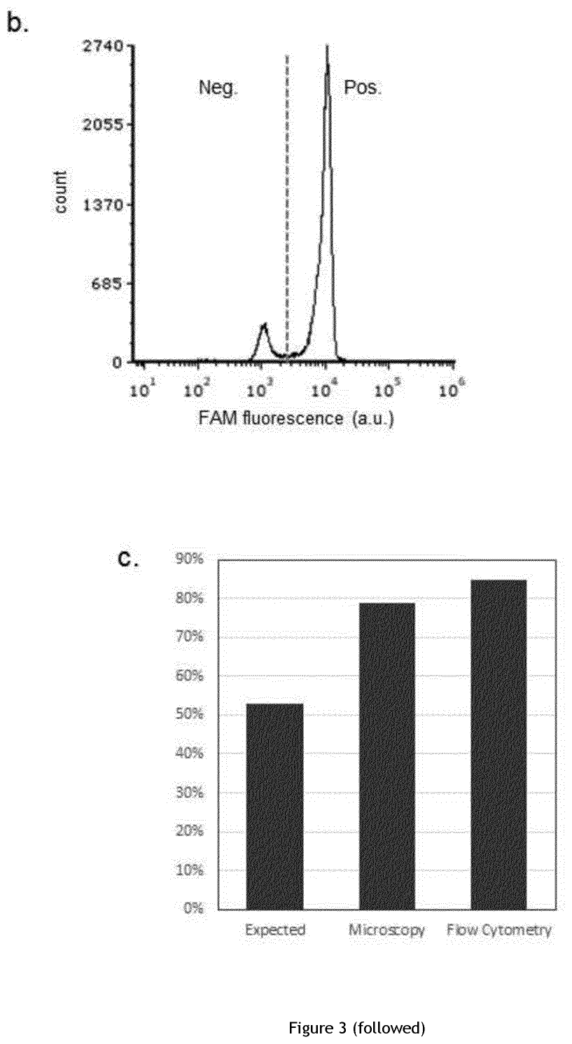

Furthermore, the inventors analyzed by flow cytometry a sample containing 1 pM of Let7a target and compare the results obtained by flow cytometry (using the on-bead rT modified with a FAM fluorophore, SEQ ID 64) and by fluorescence microscopy (using an in-solution rT modified with a Atto633 dye, SEQ ID 63). Microscopy image analysis (FIG. 3a) demonstrates that 79% of particles-containing droplets (white dots) detected the target (indicated by the gray-fluorescent droplets). These results is in good agreement with the flow cytometry readout for which 85% of positive events were detected (FIG. 3b).

### Example 2: Enzymes Trapping Effect

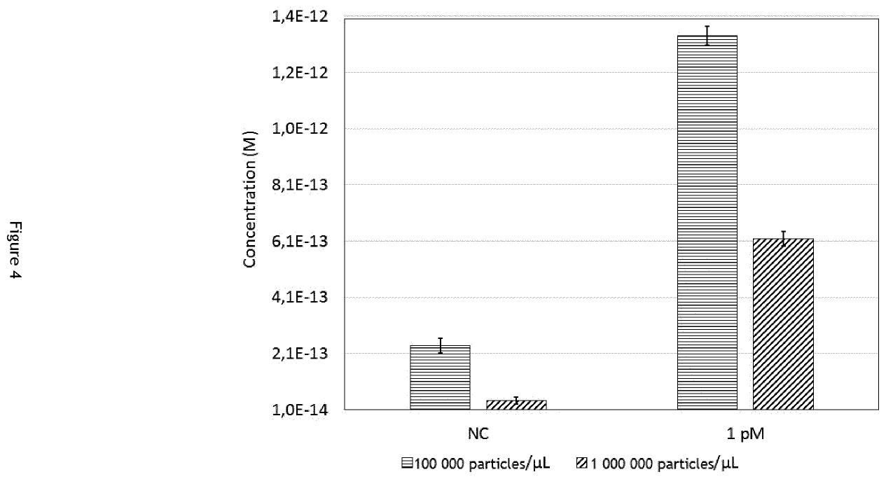

The inventors examined the effect of the particle concentration on the false positive rate. 2·10⁷ particles were incubated in presence of 0 or 1 pM or Let7a target (20 μL). The particles were then resuspended in the master mix either at 10⁵ or 10⁶ part·/μL, before being emulsified via droplet microfluidics, incubated at 50° C. for 4 hours and analyzed by flow cytometry (FIG. 4). For 10⁵ or 10⁶ part·/μL, the negative control recorded respectively 16.4% and 2.6% of false positive events, demonstrating a significant effect of the particle concentration on the sensitivity of the assay. The inventors demonstrate that pre-incubation of the particle in the master mix before partitioning leads to the concentration of the enzymes (one or several) on the DNA-grafted particle. As a result, the overconcentration of enzyme (e.g. the DNA polymerase) accelerates the non-specific amplification reaction and eventually increases the false positive rate.

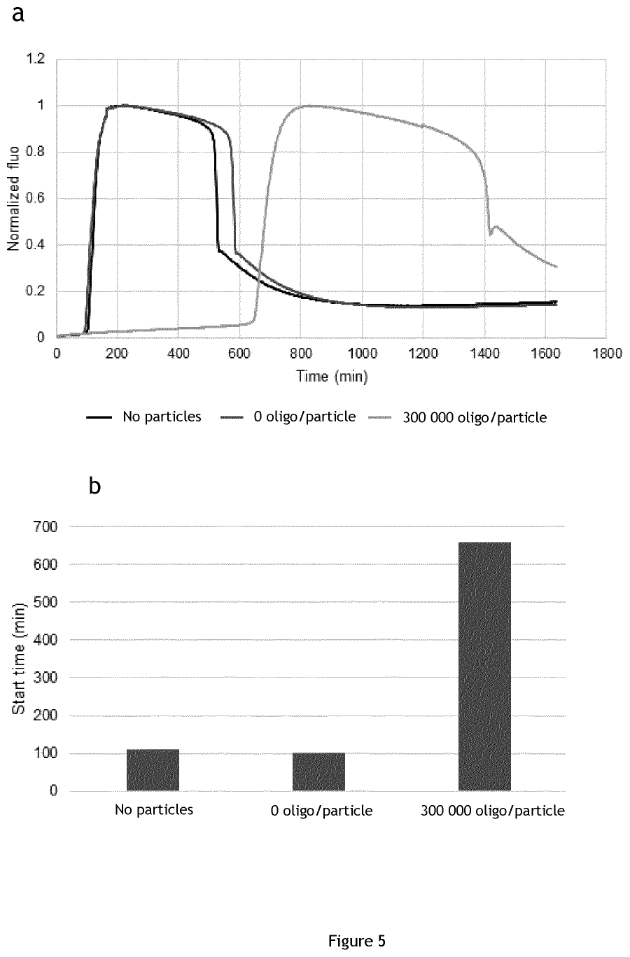

To demonstrate further this effect, the inventors designed the experiment shown in FIG. 5: an enzyme mixture (polymerase, nickases, exonuclease) used for microRNA detection is incubated for 30 minutes i) without particles, ii) with unfunctionalized particles and iii) with oligonucleotide functionalized particles. Then, the particles are pelleted and the supernatant is used as the enzyme mix for the detection of Let7a in solution: 10 pM of Let7a and the molecular program (a set of oligonucleotide including aT, SEQ ID 61 pT, SEQ ID 62 cT, SEQ ID 68, rT, SEQ ID 63) are added, and the mix is incubated at 50° C. while the fluorescence of the reporting template is monitored in real-time (FIG. 5a). FIG. 5b compares the start times (Cq) of the three samples. Using the enzyme mixed preincubated without particles or with unfunctionalized particles, the Cq are very similar (˜100 minutes), demonstrated that the particles themselves do not affect the reaction. The mixing of the enzymes with oligo-carrying particles may slow the reaction down and induce false negative.

To counter this effect and to accelerate the reaction speed and increase its sensitivity, the reaction is performed at constant particle concentration. Moreover, the particle dilution is compensated by adding a population of “neutral particles”, keeping the particle concentration constant. These neutral particles carry the same amount of reporter templates as the detection particles, but no conversion template. Hence, neutral particles trap as much enzymes as detection particles, but are unable to capture microRNAs and trigger the amplification.

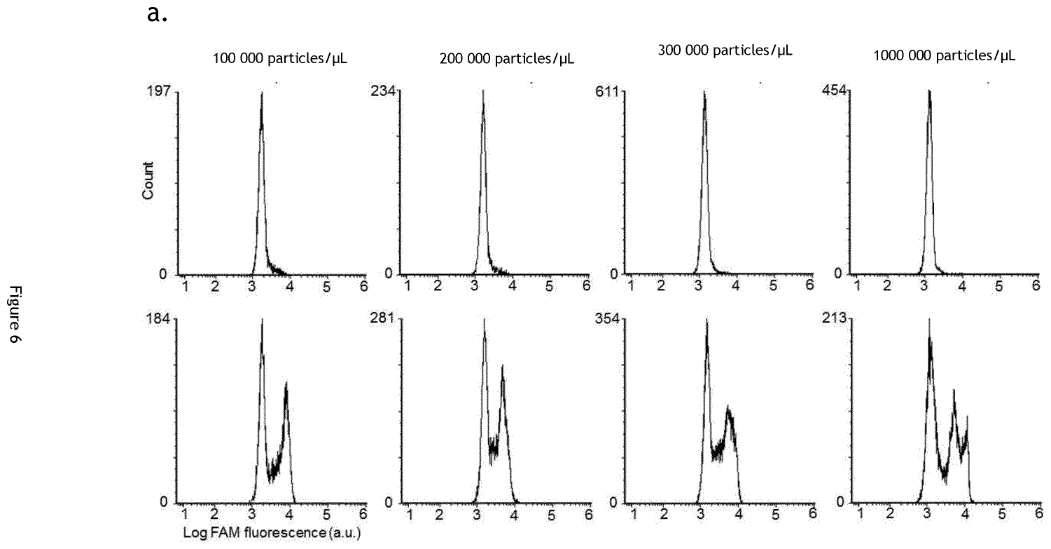

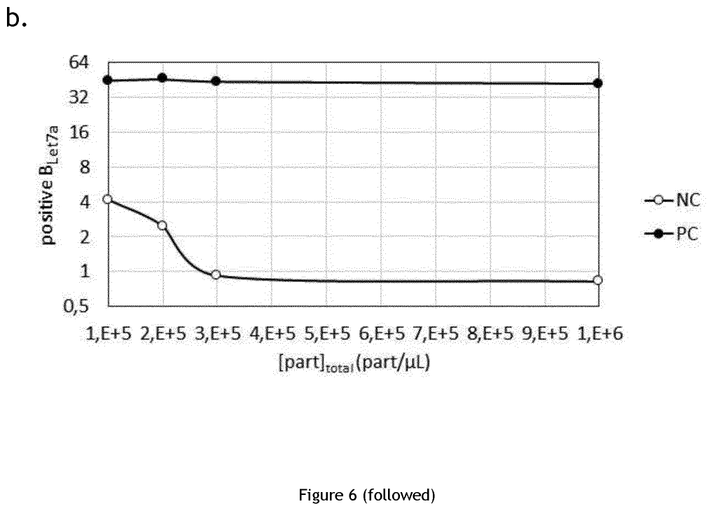

Particularly, a constant concentration of detection particles for Let7a (B_{Let7a} at 10⁵ beads/μL), supplemented with 0 to 2·10⁵ neutral particules (BN, which are not grafted with a cT) has been used. FIG. 6 shows the proportion of positive B_{Let7a} for the samples incubated with 0 or 1 pM of Let7a target. Comparing the negative controls, the inventors demonstrated that the addition of neutral beads reduces the percentage of false positive whereas it does not affect the microRNA quantification. At 10⁵ particles/μL, the reaction is complete after 4 hours. At 3·10⁵ particles/μL, it takes around 10 hours, and at 10⁶ particles/μL, the reaction is not complete after 24 hours. Finally, the inventors concluded that the optimal particles concentration may be about 3·10⁵ particles/μL.

In order to validate these experimental conditions, the inventors performed the digital detection of Let7a at different concentration. FIG. 7 shows that the false positive proportion is only 0.7%, which proves the effect of the addition of neutral particles. In the positive samples, the detected concentrations are in accordance with the expected ones. Overall, these results demonstrated that DNA-functionalized particles are suitable for the digital detection of one microRNA target.

Multiplex and Digital Detection of microRNA

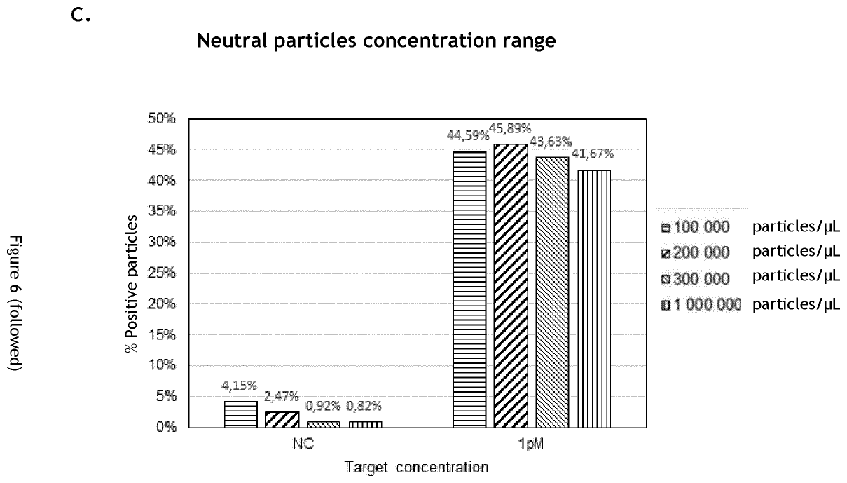

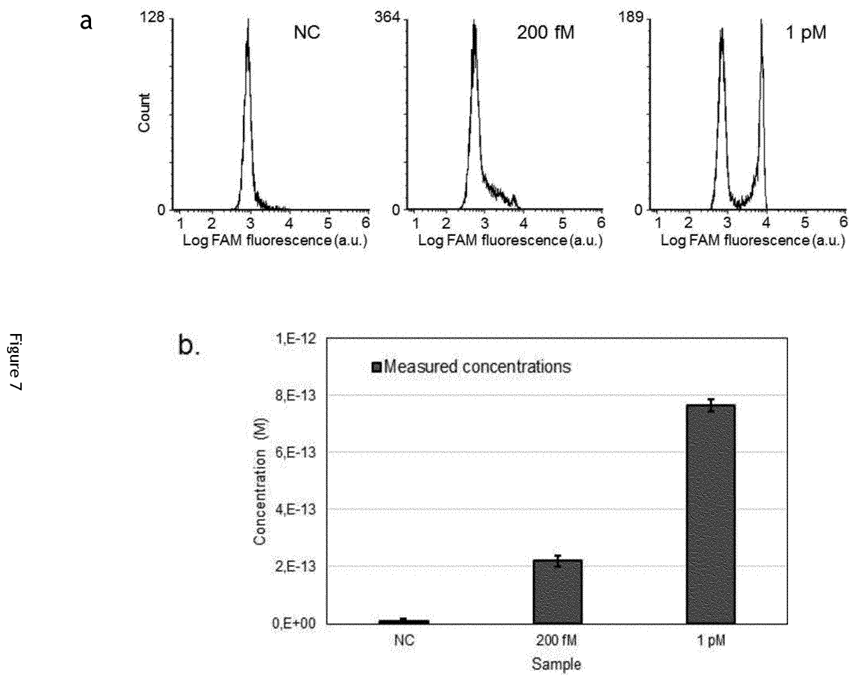

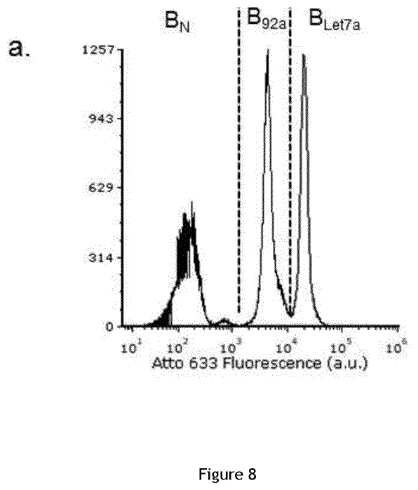

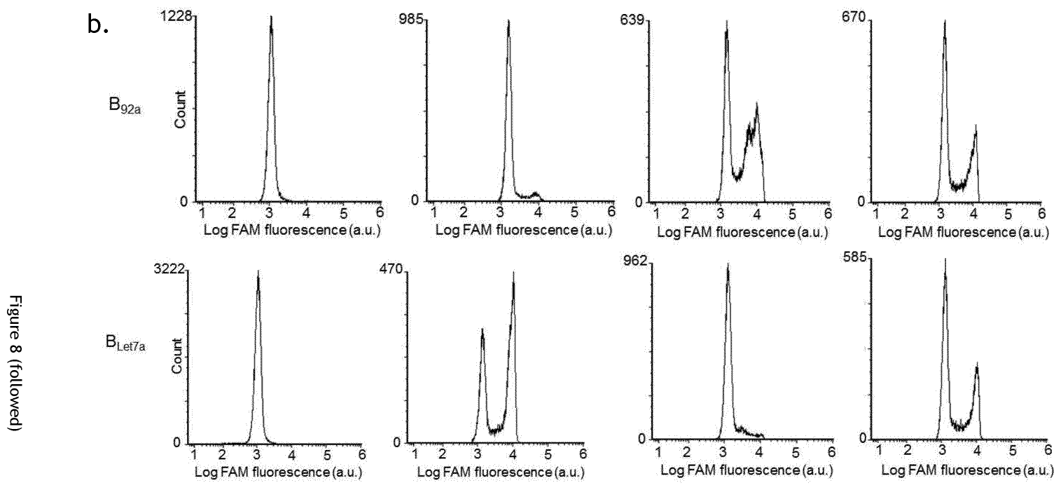

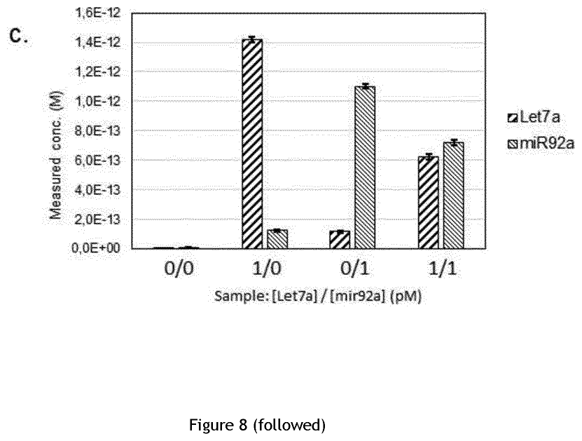

With the system well characterized for the detection of one target, the inventors proceeded to the detection of several targets in a single assay. FIG. 8 shows a duplex assay performed with two populations of particles B_{Let7a} and B₉₂ₐ, targeting respectively the microRNAs Let7a and mir92a. To be able to discriminate them, both populations are barcoded with different concentrations of a biotinylated fluorescent marker. The experiment was composed of four samples: one negative control (no target); two samples containing 1 pM of only one of the two targets; one sample containing 1 pM of both targets.

As in a single target assay, particles populations significantly turn on only if their specific target is present. The false positive percentages in the negative control are very low (0.14% for B_{Let7a}, 0.64% for B92a). It is noted that false positive percentages are higher when the other target is present. For example, in the Let7a only sample, 7% of B92a turn on. This is probably due to co-encapsulated particles, which can be mathematically compensated. The detected concentrations are in accordance with the expected ones. Hence, the inventors demonstrated here that it is effectively possible to measure two miRNAs in a single assay.

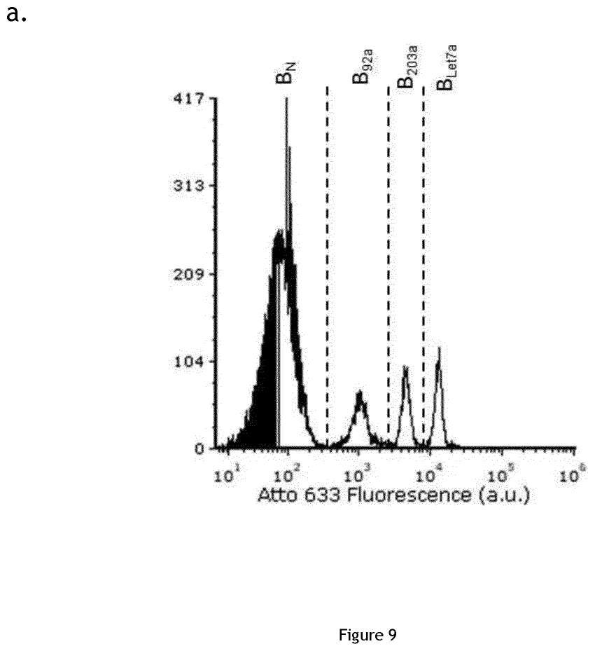

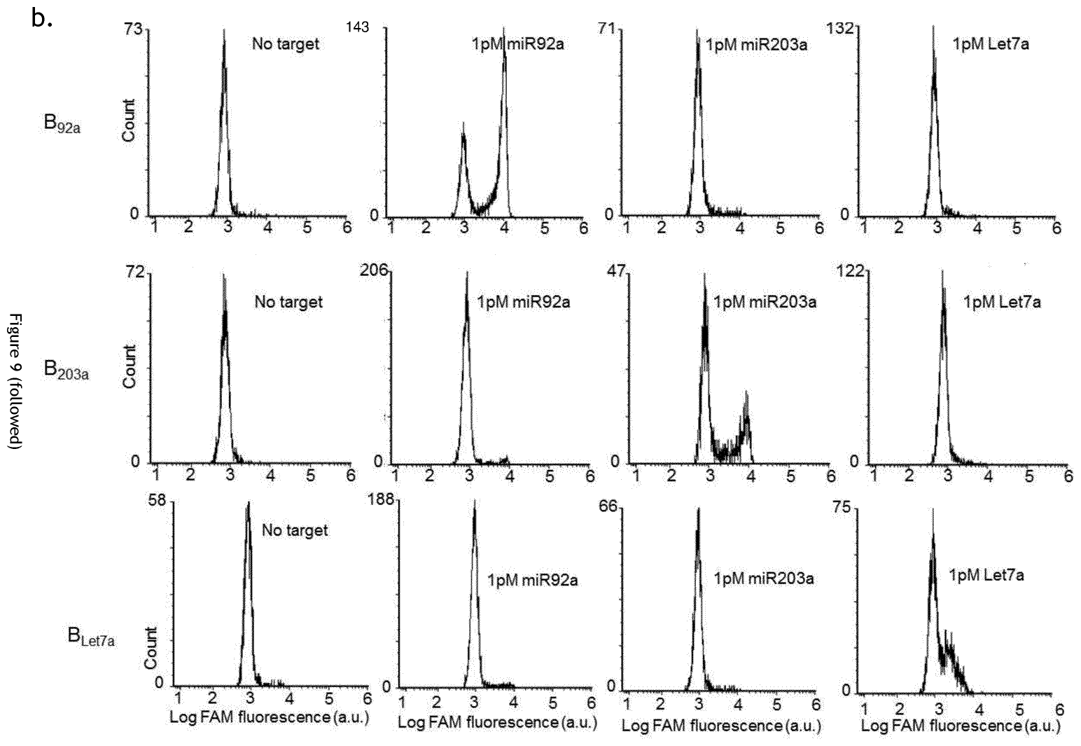

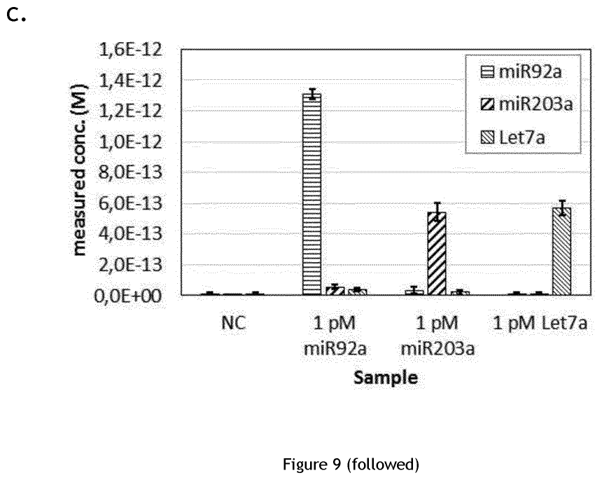

The inventors then moved on to a 3-target assay (Let7a, mir203a and mir92a) to further demonstrate the generalization of the digital multiplex approach. The cognate particles, i.e., particles corresponding to each of target biomolecule (i.e. modified with corresponding conversion template (cT), SEQ ID 66-68) plus the neutral particle population) are spiked with 1 pM of one of the three targets. The results of the flow cytometry analysis are presented in FIG. 9.

This multiplexing capability is only limited by the number of different particles populations but these one may be separated by flow cytometry for example.

This multiplexing capability is only limited by the number of different particles populations but these one may be separated by flow cytometry.

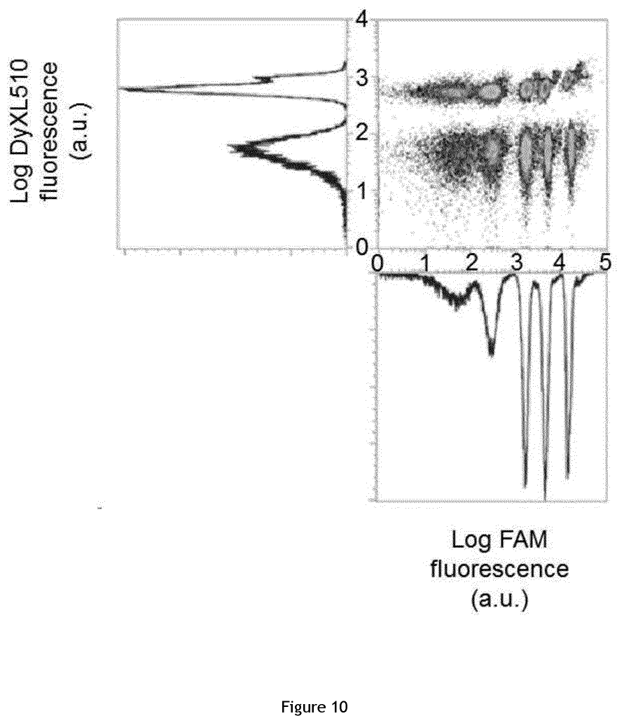

FIG. 10 demonstrates the discrimination of ten bead population using a combination of two fluorescent barcodes (with respectively five and two levels of fluorescence).

MicroRNA Detection from Biological Samples

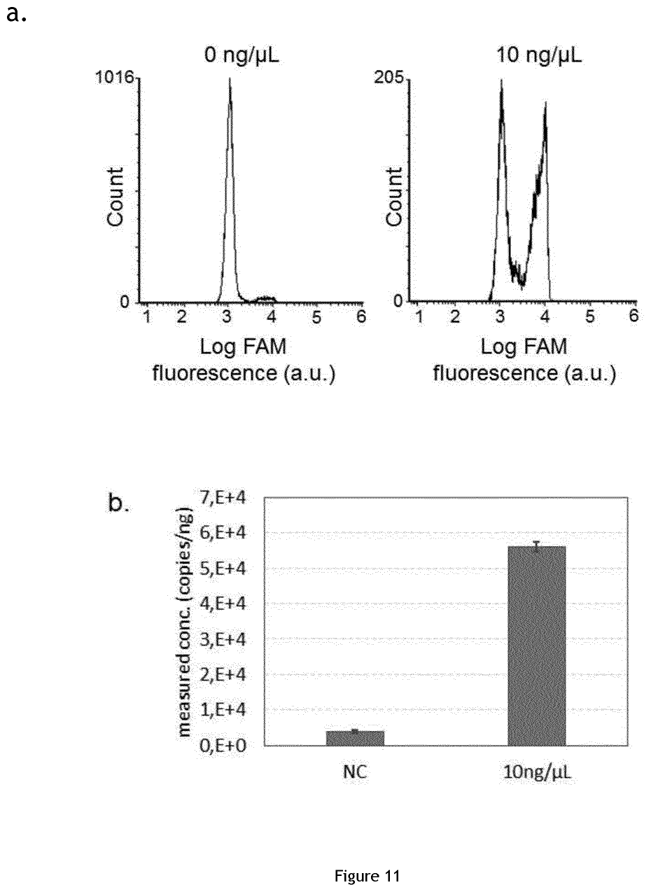

By this experiment, the inventors demonstrated the compatibility of the current digital detection approach for the detection of microRNA targets from biological samples (RNA extraction from human intestine cells). The proposed procedure decorrelates the target capture step from the signal amplification. After the capture, the particles can thus be washed and resuspended in an appropriate buffer compatible with the downstream biochemical reactions. Hence, this assay is compatible with biological samples made of complex media, the washing step enabling to get rid of the initial matrix that could interfere with the amplification reaction. Human colon total RNA, at a final concentration of 10 ng/μL is mixed with B_{Let7a} before proceeding to the quantification of the Let7a target. FIG. 11 shows the detected concentration of Let7a, approximated to 6·10⁴ copies per ng of extract. This demonstrates that the present invention allows for the detection of endogenous microRNA in biological samples.

Adjustment of the Sensitivity and Dynamic Range

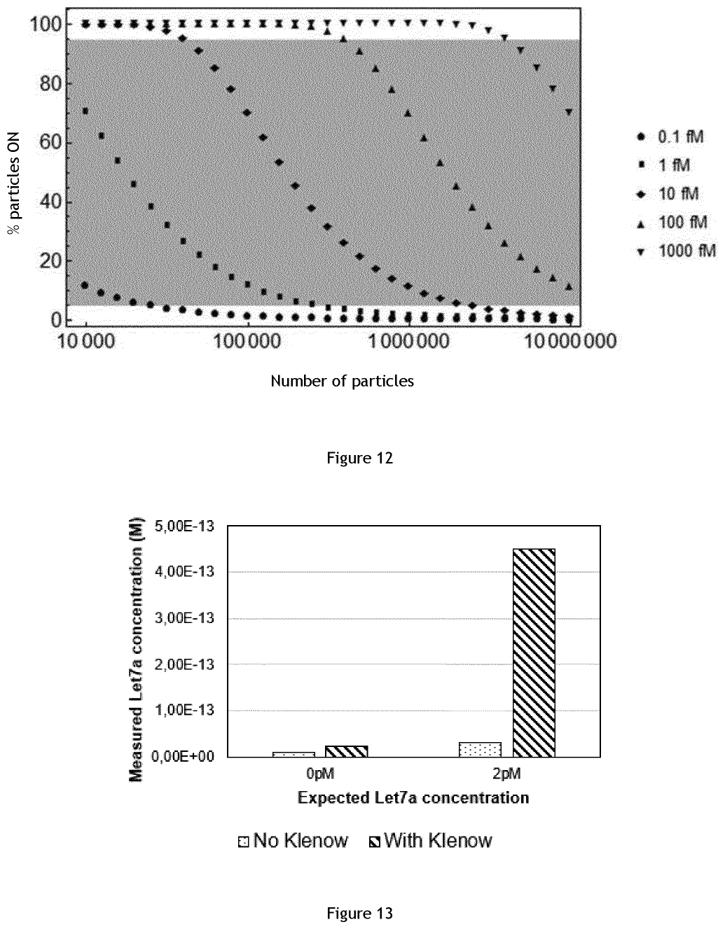

The assay sensitivity is of paramount important when considering the quantification of low abundance target. The sensitivity of a digital assay is tightly correlated to the ratio of positive/negative events, which is given by the average number of targets per particle (A). In the present assay, this parameter was adjusted by modifying the amount of detection particle. Therefore, the assay dynamic range and sensitivity can be tuned independently for each target. According to the graphic presented in FIG. 12, for highly abundant targets that are expected to saturate the particle population (for λ>5, the expected percentage of positive particle is >99%), the number of detection particles can be increased to adjust parameter λ. On the opposite, the amount of detection particles targeting poorly expressed targets can be decreased. Alternatively, for low target concentration, it is possible to increase the volume of sample used during the capture step. As a result, it will increase the number of particles hybridize to their target therefore raising the λ value.

### Example 3: Use of Two Polymerases for Nucleic Acid Detection

Further to use of (Vent(exo-)) polymerase allowing sensitive quantification of DNA synthetic targets, the inventors assessed the effect of adding Klenow(exo-) polymerase to the amplification reaction for the quantification of the microRNA. Firstly, this assay was performed for detecting Let7a.

Capture Step

miRNA capture was realized by incubating the detection particles, the miRNA target, 25 μM of dATP, dTTP, dCTP and dGTP, and Klenow polymerase at 40° C. for 2 hours. The concentrations are presented in Table 3 below:

TABLE 3
Concentrations of Klenow polymerase, particles and microRNA

| Sample | Klenow polymerase | Particles | Let7a |
|---|---|---|---|
| 1 | 0 units/mL | 10⁹ particles/mL | 0 pM |
| 2 | 0 units/mL | 10⁹ particles/mL | 2 pM |
| 3 | 50 units/mL | 10⁹ particles/mL | 0 pM |
| 4 | 50 units/mL | 10⁹ particles/mL | 2 pM |

Stirring was applied during incubation in order to avoid particle sedimentation. After incubation, particles were recovered and washed twice in storage buffer having the composition presented in Table 4 below:

TABLE 4
Composition of the storage buffer

| Storage buffer | | |
|---|---|---|
| Tris HCl pH 7.5 | 5 | mM |
| NaCl | 50 | mM |
| | 0.5 | mM |
| MgSO₄ | 5 | mM |

The reaction mixtures A and B used in the present assay are presented in Table 5 below:

TABLE 5
The composition of reaction mixtures A and B

| | Concentrations | |
|---|---|---|
| Mix A | | |
| miR Buffer | 1X | |
| Nb Bsml | 400 | u/mL |
| Vent (exo−) | 160 | u/mL |
| Nt.Bst.NBI | 20 | u/mL |
| BSA | 400 | μg/mL |
| ttRecJ/140 | 26 | nM |
| Bsml | 100 | u/mL |
| Mix B | | |
| miR Buffer | 1X | |
| CBc12-2PS4 | 100 | nM |
| pTBc12T5SP | 16 | nM |
| rTBc | 100 | nM |
| Particles | 5.10⁸ | particles/mL |

Encapsulation

Mix A and Mix B are encapsulated in 50%/50% proportion in 9 μm water-in-oil droplets using a double water inlet flow focusing microfluidic device.

Incubation

The droplets are incubated at 50° C. for 8 hours.

Particle Analysis

The droplets are broken using an electrostatic gun (Zerostat 3, Milty, UK). The particles are resuspended in Attune N×T focusing fluid (ThermoFisher) and analysed by flow cytometry (Attune N×T flow cytometer, ThermoFisher).

FIG. 13 shows that the introduction of Klenow(exo-) during the capture step (i. the capture of miRNA (Let7a) on the particle surface) clearly increases the detected amount of Let7a: With Klenow(exo-) in the capture mixture, 450 fM of Let7a were detected, whereas only 31 fM were detected if there was no Klenow(exo-) during the capture step.

For optimizing the action of Klenow (exo-) polymerase and for eliminating the remaining background amplification (due to the unspecific amplification), if any, the inventors enhanced the experimental protocol described above by performing additional steps of post-capture washing in a stringent buffer and sonication steps in order to remove trapped polymerase molecules.

Capture Step

miRNA capture was realized by incubating the detection particles, the miRNA target and Klenow polymerase at 40° C. for 2 hours, under agitation to avoid particle sedimentation (2000 rpm). The concentrations were:
Particles: 10⁹ particles/mL
Klenow polymerase: 50 units/mL

Post-Capture Washing:

“Soft” Washing Procedure:
Particles are magnetically pooled and the rest of the capture mix is discarded
Resuspension in storage buffer
Vortex 30 s
Supernatant is discarded
Resuspension in storage buffer
Vortex 30 s
Supernatant is discarded
Resuspension in storage buffer
Vortex 30 s

“Hard” Washing Procedure:
Particles are magnetically pooled and the rest of the capture mix is discarded
Resuspension in BW buffer
Vortex 30 s
Ultrasound bath sonication 30 s
Supernatant is discarded
Resuspension in BW buffer
Vortex 30 s
Ultrasound bath sonication 30 s
Supernatant is discarded
Resuspension in storage buffer
Vortex 30 s
Supernatant is discarded
Resuspension in storage buffer
Vortex 30 s
Supernatant is discarded
Resuspension in storage buffer
Vortex 30 s

The storage buffer has the same composition as the one shown in Table 4 above. The composition of BW buffer is presented in Table 6 below:

TABLE 6
Composition of BW buffer

| BW buffer | |
|---|---|
| Tris-HCl pH 7.5 | 20 mM |
| NaCl | 1M |
| EDTA | 1 mM |
| Tween 20 | 0.2% |

The reaction mixtures A and B have the same composition as those presented in Table 5 above. Moreover, the encapsulation, the incubation and the particle analysis are performed at the same manner as described above.

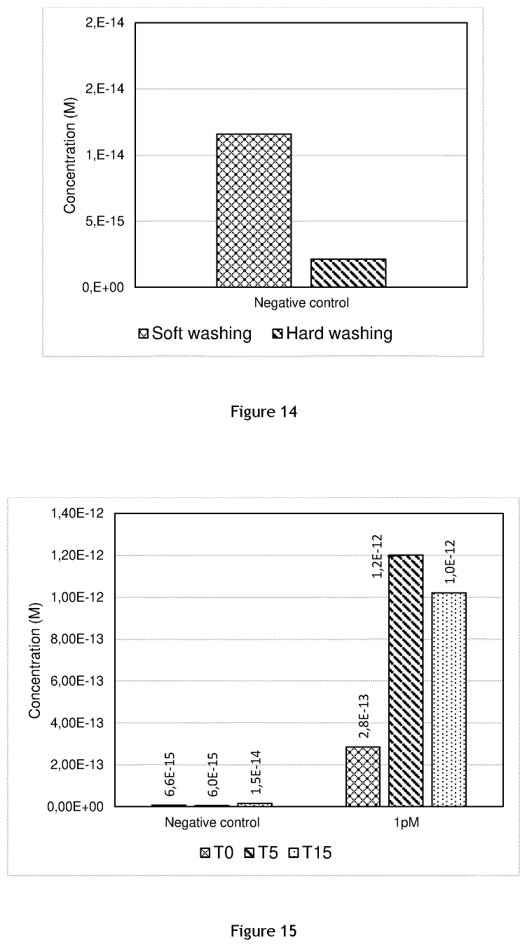

FIG. 14 shows that the detected concentration from the negative control (sample without biomolecule target) is reduced by 80% thanks to the “hard” washing procedure. This demonstrates that Klenow polymerase molecules is trapped on the particles and that the “hard” washing procedure allows to eliminate unspecific amplification (background amplification).

### Example 4: Designing the Conversion Template as Poly(T) Conversion Template

As demonstrated above, the addition of Klenow polymerase to the capture step increased the detected amount of RNA targets. In order to further improve the sensitivity of the method of the present multiplex method, the inventors designed conversion templates comprising a poly(T) spacer in between the microRNA binding sequence and the Nt.BstNBI recognition site, thus increasing the number of DNA nucleotides on the primer.

The experimental conditions (capture step, post-capture washing (hard wash protocol), encapsulation, incubation and particle analysis) are as those described above. It is however noted that only dATP (25 μM) is introduced during the capture step.

I

FIG. 15 shows that T5 and T15 (corresponding to a poly (T) spacer of 5 to 15 nucleotides of length, which corresponds to sequence 21 toBc T5 T7 biot (for T5) and 21 toBc T15 T7 biot (for T15)); converter templates allow an accurate quantification of miR21: The detected concentrations are 1.20 pM and 1.02 pM, respectively, whereas the original TO cT only detects 0.28 pM. Moreover, the T5 converter does not increase the background amplification.

### Example 5: 6-Plex Detection of Synthetic miRNA

With this set of optimized conditions (Klenow polymerase in capture, “hard” washing procedure, T5 converter template) the inventors assessed the detection of synthetic miRNAs spiked in water.

Capture Step

miRNA capture was performed by incubating the detection particles, the miRNA target and Klenow polymerase at 40° C. for 2 hours. The concentrations were:

Particles: 2.5·10⁸ particles/mL for each of the 6 subpopulations (1.5·10⁹ particles/mL total)
Klenow polymerase: 50 units/mL

The concentrations of six miRNA are presented in Table 7 below:

TABLE 7
Concentrations of miRNA

| Target | Concentration | |
|---|---|---|
| Let7a | 500 | fM |
| miR21 | 10 | fM |
| | 500 | fM |
| miR203a | 10 | fM |
| miR16 | 500 | fM |
| miR10a | 10 | fM |

Stirring was applied during incubation in order to avoid particle sedimentation.

The other experimental conditions (capture step, post-capture washing (hard wash protocol), encapsulation, incubation and particle analysis) are as those described above. It is however noted that only dATP (25 μM) is introduced during the capture step.

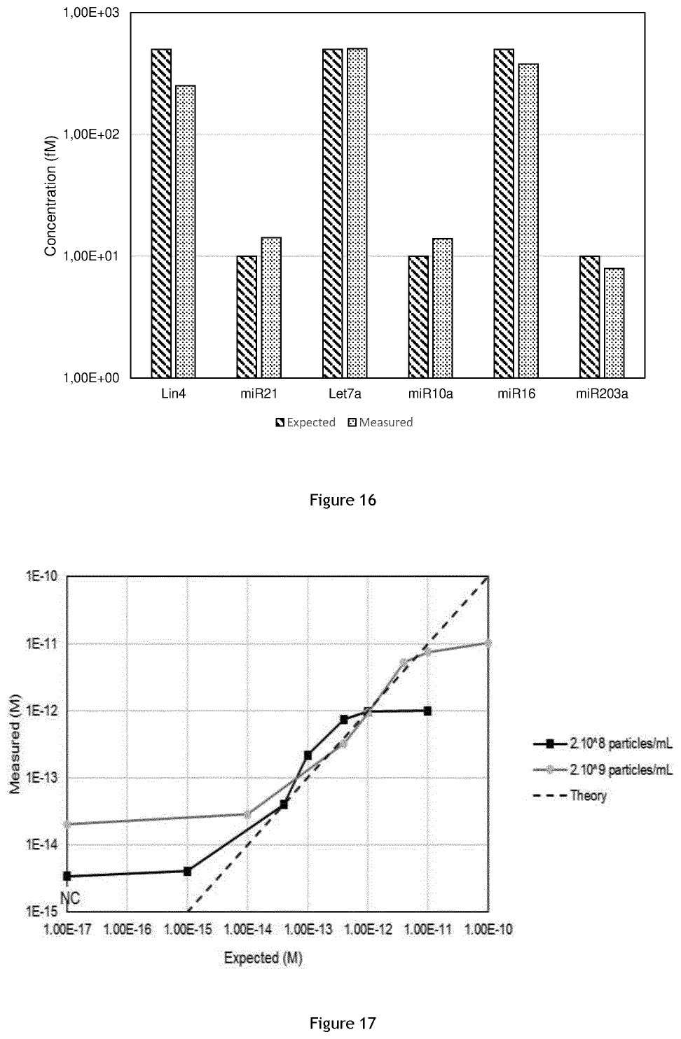

FIG. 16 shows that the measured pattern is very close to the expected one (1.00 E-12). The new experimental conditions allow the multiplex quantification of RNA targets, which could be applied to the detection of disease-associated molecular signatures.

### Example 6: Tunable Dynamic Range

The detected concentration of microRNA target is given by the equation:

[microRNA]=−ln (1−Fₚₒₛ)·[Particles]_{Capture}

Where Fₚₒₛ is the percentage of positive particles, comprised between 0% and 100%. The very high variability of the measured miRNA concentration for extreme values of Fₚₒₛ (close to 0% or 100%) decreases the reliability of the quantification at such Fₚₒₛ values. The dynamic range, in which the miRNA target can be reliably quantified, is thus limited.

It is however possible to broaden the dynamic range by adapting the concentration of particles during the capture step. Lowering the concentration of detection particles allows to detect the target at lower concentrations.

In order to assess the dynamic range of the test and to verify that it can be modified by changing the particles concentration, various amounts of Let7a are quantified using 2 different concentrations of particles.

Capture Step

miRNA capture was realized by incubating the detection particles, the miRNA target and Klenow polymerase at 40° C. for 2 hours. The concentrations were:
Klenow polymerase: 50 units/mL in all samples
Buffer: miR buffer 1× dATP only

TABLE 8
Concentration of miRNA

| Sample | [Particles] | [Let7a] | |
|---|---|---|---|
| 1 | 2.10⁹ particles/mL | 0 | fM |
| 2 | 2.10⁹ particles/mL | 10 | fM |
| 3 | 2.10⁹ particles/mL | 400 | fM |
| 4 | 2.10⁹ particles/mL | 1 | pM |
| 5 | 2.10⁹ particles/mL | 4 | pM |
| 6 | 2.10⁹ particles/mL | 10 | pM |
| 7 | 2.10⁹ particles/mL | 100 | pM |
| 8 | 2.10⁸ particles/mL | 0 | fM |
| 9 | 2.10⁸ particles/mL | 1 | fM |
| 10 | 2.10⁸ particles/mL | 40 | fM |
| 11 | 2.10⁸ particles/mL | 100 | fM |
| 12 | 2.10⁸ particles/mL | 400 | fM |
| 13 | 2.10⁸ particles/mL | 1 | pM |
| 14 | 2.10⁸ particles/mL | 10 | pM |

After the capturing step a hard washing was performed as described above.

Reaction Mixtures:

TABLE 9
Reaction mixtures and concentrations

| | Concentrations | |
|---|---|---|
| Mix A | | |
| miR Buffer | 1X | |
| Nb Bsml | 400 | u/mL |
| Vent (exo−) | 160 | u/mL |
| Nt.Bst.NBI | 20 | u/mL |
| BSA | 400 | μg/mL |
| ttRecJ/140 | 26 | nM |
| Bsml | 100 | u/mL |
| Bsml | 1% | v/v |
| Mix B | | |
| miR Buffer | 1X | |
| CBc12-2PS4 | 100 | nM |
| pTBc12T5SP | 16 | nM |
| MBBc.Bsml.Atto633.BHQ2 | 100 | nM |
| Particles | 5.10⁸ | particles/mL |

Encapsulation

Mix A and Mix B are encapsulated in 50%/50% proportion in 9 μm water-in-oil droplets using a double water inlet flow focusing microfluidic device.

Incubation

The droplets are incubated at 50° C. for 8 hours.

Particle Analysis:

The droplets are broken using an electrostatic “gun”. The particles are resuspended in Attune N×T focusing fluid (ThermoFisher) and analysed by flow cytometry (Attune N×T flow cytometer, Thermo Fisher).

FIG. 17 shows that for a concentration of detection particles in the capture mix of 2·10⁹ particles/mL, the dynamic range is comprised between 10 pM and 100 fM. Reducing the concentration of particles to 2·10⁸ particles/mL shifts the dynamic range by approximately one order of magnitude (1 pM-10 fM).

### Example 7: Multiplex Detection from Human Colon Total RNA

The system is working on synthetic miRNA targets spiked in water. Here the inventors demonstrate the multiplex detection of 3 microRNA targets in human colon total RNA. In this assay, the inventors detected human miRNAs Let7a, miR21 while Lin4 from Caenorhabditis elegans as a control.

Capture Step

miRNA capture was realized by incubating the detection particles, the miRNA target and Klenow polymerase at 40° C. for 2 hours. The concentrations were:
Klenow polymerase: 50 units/mL in all samples
Particles: 5·10⁸ particles/mL for each of the 3 subpopulations (1.5·10⁹ particles/mL total)
Total RNA: 2.5 μg/mL
Lin4: 1 pM (exogenous spike-in control)
Buffer: miR buffer 1×dATP only

After the capture step a hard washing was performed as described above.

Reaction Mixtures:

The reaction mixtures A and B were the same (same composition and same concentrations) as those shown in Table 9 above.

Moreover, the encapsulation, the incubation and the particle analysis were performed as described on Example 6.

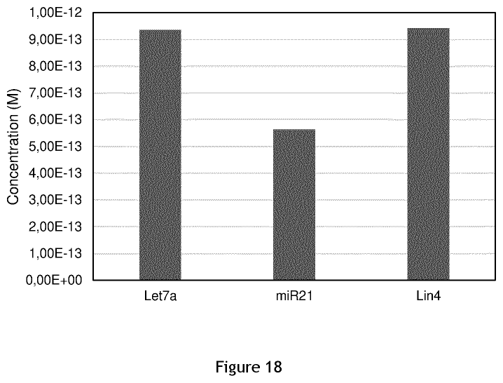

FIG. 18 shows that the system successfully detected 3 microRNAs from a sample of human colon total RNA, including two endogenous targets (let7a and mir21) and 1 exogenous target (spike-in lin4). The expected concentration of control microRNA lin4 was 1 pM and the measured concentration is 0.94 pM.

### BIBLIOGRAPHICAL REFERENCES

[1] S. Roy, J. H. Soh, and J. Y. Ying, “A microarray platform for detecting disease-specific circulating miRNA in human serum,” Biosensors and Bioelectronics, vol. 75, pp. 238-246, January 2016.
[2] T. Ueno and T. Funatsu, “Label-Free Quantification of MicroRNAs Using Ligase-Assisted Sandwich Hybridization on a DNA Microarray,” PLOS ONE, vol. 9, no. 3, p. e90920, March 2014.
[3] Y. Sun et al., “Development of a micro-array to detect human and mouse microRNAs and characterization of expression in human organs,” Nucl. Acids Res., vol. 32, no. 22, pp. e188-e188, January 2004.
[4] P. T. Nelson, D. A. Baldwin, L. M. Scearce, J. C. Oberholtzer, J. W. Tobias, and Z. Mourelatos, “Microarray-based, high-throughput gene expression profiling of microRNAs,” Nat Meth, vol. 1, no. 2, pp. 155-161, November 2004.
[5] I. Beuvink et al., “A novel microarray approach reveals new tissue-specific signatures of known and predicted mammalian microRNAs,” Nucl. Acids Res., vol. 35, no. 7, p. e52, January 2007.
[6] P. Campomenosi et al., “A comparison between quantitative PCR and droplet digital PCR technologies for circulating microRNA quantification in human lung cancer,” BMC Biotechnology, vol. 16, p. 60, 2016.
[7] C. Chen et al., “Real-time quantification of microRNAs by stem-loop RT-PCR,” Nucleic Acids Res, vol. 33, no. 20, p. e179, 2005.
[8] V. Benes and M. Castoldi, “Expression profiling of microRNA using real-time quantitative PCR, how to use it and what is available,” Methods, vol. 50, no. 4, pp. 244-249, April 2010.
[9] G. Wan, Q. 'En Lim, and H. P. Too, “High-performance quantification of mature microRNAs by real-time RT-PCR using deoxyuridine-incorporated oligonucleotides and hemi-nested primers,” RNA, vol. 16, no. 7, pp. 1436-1445, January 2010.
[10] K. L. Opel, D. Chung, and B. R. McCord, “A Study of PCR Inhibition Mechanisms Using Real Time PCR*,†,” Journal of Forensic Sciences, vol. 55, no. 1, pp. 25-33.
[11] J. Aslanzadeh, “Preventing PCR Amplification Carryover Contamination in a Clinical Laboratory,” Ann Clin Lab Sci, vol. 34, no. 4, pp. 389-396, January 2004.
[12] C. A. Raabe, T. H. Tang, J. Brosius, and T. S. Rozhdestvensky, “Biases in small RNA deep sequencing data,” Nucleic Acids Res, vol. 42, no. 3, pp. 1414-1426, February 2014.
[13] S. Bustin et al., “Variability of the Reverse Transcription Step: Practical Implications,” Clinical Chemistry, vol. 61, no. 1, pp. 202-212, January 2015.
[14] P. Zhang, Y. Liu, Y. Zhang, C. Liu, Z. Wang, and Z. Li, “Multiplex ligation-dependent probe amplification (MLPA) for ultrasensitive multiplexed microRNA detection using ribonucleotide-modified DNA probes,” Chem. Commun., vol. 49, no. 85, pp. 10013-10015, October 2013.
[15] P. Zhang, J. Zhang, C. Wang, C. Liu, H. Wang, and Z. Li, “Highly Sensitive and Specific Multiplexed MicroRNA Quantification Using Size-Coded Ligation Chain Reaction,” Anal. Chem., vol. 86, no. 2, pp. 1076-1082, January 2014.
[16] V. Taly et al., “Multiplex Picodroplet Digital PCR to Detect KRAS Mutations in Circulating DNA from the Plasma of Colorectal Cancer Patients,” Clinical Chemistry, vol. 59, no. 12, pp. 1722-1731, January 2013.
[17] E. Tan et al., “Specific versus nonspecific isothermal DNA amplification through thermophilic polymerase and nicking enzyme activities,” Biochemistry, vol. 47, no. 38, pp. 9987-9999, September 2008.
[18] T. Sano, C. L. Smith, and C. R. Cantor, “Immuno-PCR: very sensitive antigen detection by means of specific antibody-DNA conjugates,” Science, vol. 258, no. 5079, pp. 120-122, October 1992.
[19] V. Taly, D. Pekin, A. E. Abed, and P. Laurent-Puig, “Detecting biomarkers with microdroplet technology,” Trends in Molecular Medicine, vol. 18, no. 7, pp. 405-416, July 2012.
[20] Y. Rondelez et al., “Microfabricated arrays of femtoliter chambers allow single molecule enzymology,” Nat Biotech, vol. 23, no. 3, pp. 361-365, March 2005.
[21] Y. Zhao, Y. Cheng, L. Shang, J. Wang, Z. Xie, and Z. Gu, “Microfluidic Synthesis of Barcode Particles for Multiplex Assays,” Small, vol. 11, no. 2, pp. 151-174, January 2015.
[22] A. Sarkar, H. W. Hou, A. E. Mahan, J. Han, and G. Alter, “Multiplexed Affinity-Based Separation of Proteins and Cells Using Inertial Microfluidics,” Scientific Reports, vol. 6, p. 23589, March 2016.
[23] S. C. Chapin and P. S. Doyle, “Ultrasensitive Multiplexed MicroRNA Quantification on Encoded Gel Microparticles Using Rolling Circle Amplification,” Anal. Chem., vol. 83, no. 18, pp. 7179-7185, September 2011.
[24] D. C. Pregibon, M. Toner, and P. S. Doyle, “Multifunctional Encoded Particles for High-Throughput Biomolecule Analysis,” Science, vol. 315, no. 5817, pp. 1393-1396, March 2007.
[25] D. S. Zhang et al., “Dual-Encoded Microbeads through a Host-Guest Structure: Enormous, Flexible, and Accurate Barcodes for Multiplexed Assays,” Advanced Functional Materials, vol. 26, no. 34, pp. 6146-6157, September 2016.
[26] Brouzes et al. “Droplet microfluidic technology for single-cell high-throughput screening”, PNAS 2009
[27] Genot et al, “High-resolution mapping of bifurcations in nonlinear biochemical circuits”, Nat. Chem. 2016.
[28] A. Yamagata, R. Masui, Y. Kakuta, S. Kuramitsu and K. Fukuyama; Overexpression, purification and characterization of RecJ protein from Thermus thermophilus HB8 and its core domain; Nucleic Acids Res. Nov. 15, 2001; 29(22): 4617-4624.
[29] Xu et al 2005: Generation of Monodisperse Particles by Using Microfluidics: Control over Size, Shape, and Composition, GDCH, Vol. 44, pages 724-728.
[30] Huggett et al. BMC Res Notes 2008
[31] Wil A. M. Loenen and Elisabeth A. Raleigh The other face of restriction: modification-dependent enzymes; Nucleic Acids Res. Jan. 1, 2014; 42(1): 56-69.

1. A digital multiplex method for detecting and/or quantifying multiple target biomolecules in a sample, comprising the following steps:
a) functionalizing a suspension of particles with one or more oligonucleotides selected from a first oligonucleotide which is a conversion oligonucleotide (cT), a second oligonucleotide which is a reporting oligonucleotide (rT), a third oligonucleotide which is an amplification oligonucleotide (aT), and a fourth oligonucleotide which is a leak absorption oligonucleotide (pT);
b) adding to the particles functionalized in step a) barcodes allowing the discrimination of the particles targeting multiple biomolecules;
c) contacting the particles obtained in step b) with a tested sample to capture the multiple target biomolecules;
d) resuspending the particles having captured or not the target biomolecules in a common amplification mixture including a buffer, enzymes, deoxy-nucleoside triphosphate (dNTPs) and optionally oligonucleotides;
e) separating the particles in the suspension obtained in step d) from each other so that each particle can react independently;
f) incubating the particles at a constant temperature so that each target biomolecule triggers an amplification reaction which generates an amplification signal on the particle carrying the target, and
g) detecting and/or measuring the signals of the particles including the barcode signal and the amplification signal of each particle.

2. The digital multiplex method of claim 1, wherein the functionalization of the suspension of particles in step a) is performed with the first oligonucleotide and with the second oligonucleotide, and
wherein the third oligonucleotide and the fourth oligonucleotide are added in the amplification mixture in step d).

3. The digital multiplex method of claim 1, wherein steps a) and b) are performed concomitantly.

4. The digital multiplex method of claim 1, further comprising a step e1) of recovering the particles.

5. The digital multiplex method according claim 1, wherein the enzymes used in step d) are selected from the group consisting of polymerase, nicking enzyme or restriction enzyme, and exonuclease.

6. The digital multiplex method according to claim 1, wherein the suspension obtained in step d) is separated in step e) into droplets.

7. The digital multiplex method according to claim 1, wherein the constant temperature in step f) is between 30 and 55° C.

8. The digital multiplex method according to claim 1, wherein the functionalized particles are selected from porous or non-porous particles and hydrogel particles having a size between 10 nm and 500 μm.

9. The digital multiplex method according claim 1, wherein the step g) of detecting and/or measuring said barcode signal comprises detecting and/or measuring the barcode signal for each particle associated to the target biomolecule and the signal resulting from the amplification.

10. The digital multiplex method of claim 1, wherein the target biomolecules are of the same kind or of different kind, said biomolecules being nucleic acids or proteins.

11. The digital multiplex method according to claim 10, wherein the target biomolecules are nucleic acids selected from the group consisting of DNAs, cDNAs, RNAs, mRNAs, and microRNAs.

12. The digital multiplex method according to claim 1, wherein the target biomolecule is used as a biomarker.

13. An in vitro method for diagnosis of a disease selected from the group consisting of cancer, neuronal diseases, cardiovascular diseases, inflammatory diseases, autoimmune diseases, diseases due to a viral or bacterial infection, skin diseases, skeletal muscle diseases, dental diseases, and prenatal diseases comprising the use of the digital multiplex method according to claim 1.

14. An in vitro method for agro diagnosis of a disease selected from the group comprising:
diseases caused by biotic stress, or
diseases caused by abiotic stress,
said method comprising the use of the multiplex digital method according to claim 1.

15. A kit for detecting and/or quantifying multiple target biomolecules comprising:
a) a suspension of particles functionalized with a one or more oligonucleotides selected from a first oligonucleotide which is a conversion oligonucleotide (cT), a second oligonucleotide which is a reporting oligonucleotide (rT), a third oligonucleotide which is an amplification oligonucleotide (aT), and a forth oligonucleotide which is a leak absorption oligonucleotide (pT), to which particles are added different barcodes allowing the discrimination of the particles targeting different biomolecules;
b) a mixture of enzymes, and
c) a separating agent.

16. The digital multiplex method of claim 1, wherein the particles are microparticles.

17. The digital multiplex method according to claim 6, wherein the droplets are water-in-oil emulsion droplets having a size of droplet is comprised between 0.001 and 100 pL.

18. The digital multiplex method according to claim 9, wherein said barcode signal is a fluorescence signal.

19. The in vitro method for agro diagnosis according to claim 14, wherein:
the diseases caused by biotic stress are from infectious and/or parasitic origin, or
the diseases caused by abiotic stress are caused by nutritional deficiencies and/or unfavorable environment,

20. The kit according to claim 15, wherein the particles are microparticles and the enzymes of said mixture are selected from the group consisting of polymerase, nicking enzyme or restriction enzyme, and exonuclease.


SEQUENCE LISTING (82 sequences)

SEQ ID NO: 1 - DNA, 24 bp, Artificial Sequence
  CBe12PS3autocatalytic template
cgatcctgaatgcgatcctgaatg

SEQ ID NO: 2 - DNA, 23 bp, Artificial Sequence
  CBe12-1PS3autocatalytic template
cgatcctgaatgcgatcctgaat

SEQ ID NO: 3 - DNA, 22 bp, Artificial Sequence
  CBe12-2PS3autocatalytic template
cgatcctgaatgcgatcctgaa

SEQ ID NO: 4 - DNA, 17 bp, Artificial Sequence
  ptBe12T5SPpseudotemplate
tttttcgatcctgaatg

SEQ ID NO: 5 - DNA, 23 bp, Artificial Sequence
  CBa12-1PS4autocatalytic template
ctcgtcagaatgctcgtcagaat

SEQ ID NO: 6 - DNA, 16 bp, Artificial Sequence
  ptBa12A4SPpseudotemplate
aaaactcgtcagaatg

SEQ ID NO: 7 - DNA, 17 bp, Artificial Sequence
  ptBa12T5SP pseudotemplate
tttttctcgtcagaatg

SEQ ID NO: 8 - DNA, 16 bp, Artificial Sequence
  ptBa12T4S3Ppseudotemplate
ttttctcgtcagaatg

SEQ ID NO: 9 - DNA, 15 bp, Artificial Sequence
  ptBa12T3S3Ppseudotemplate
tttctcgtcagaatg

SEQ ID NO: 10 - DNA, 14 bp, Artificial Sequence
  ptBa12T2S3Ppseudotemplate
ttctcgtcagaatg

SEQ ID NO: 11 - DNA, 13 bp, Artificial Sequence
  ptBa12T1S3Ppseudotemplate
tctcgtcagaatg

SEQ ID NO: 12 - DNA, 22 bp, Artificial Sequence
  CBa12-2PS4autocatalytic template
ctcgtcagaatgctcgtcagaa

SEQ ID NO: 13 - DNA, 24 bp, Artificial Sequence
  CBa12PS4autocatalytic template
ctcgtcagaatgctcgtcagaatg

SEQ ID NO: 14 - DNA, 23 bp, Artificial Sequence
  CBa12-1PS4autocatalytic template
ctcgtcagaatgctcgtcagaat

SEQ ID NO: 15 - DNA, 21 bp, Artificial Sequence
  CBa12-3PS4autocatalytic template
ctcgtcagaatgctcgtcaga

SEQ ID NO: 16 - DNA, 17 bp, Artificial Sequence
  pTk12T5S4Ppseudotemplate
  misc_feature (13)..(13): n is u
tttttcaatgacncctg

SEQ ID NO: 17 - DNA, 14 bp, Artificial Sequence
  ApTk12A1SUPpseudotemplate
  misc_feature (9)..(9): n is u
acaatgacncctga

SEQ ID NO: 18 - DNA, 15 bp, Artificial Sequence
  ApTk12A2SUPpseudotemplate
  misc_feature (10)..(10): n is u
aacaatgacncctga

SEQ ID NO: 19 - DNA, 16 bp, Artificial Sequence
  ApTk12A3PSpseudotemplate
  misc_feature (11)..(11): n is u
aaacaatgacncctga

SEQ ID NO: 20 - DNA, 17 bp, Artificial Sequence
  ApTk12A4SUPpseudotemplate
  misc_feature (12)..(12): n is u
aaaacaatgacncctga

SEQ ID NO: 21 - DNA, 18 bp, Artificial Sequence
  ApTk12A5SUPpseudotemplate
  misc_feature (13)..(13): n is u
aaaaacaatgacncctga

SEQ ID NO: 22 - DNA, 19 bp, Artificial Sequence
  ApTk12A6SUPpseudotemplate
  misc_feature (14)..(14): n is u
aaaaaacaatgacncctga

SEQ ID NO: 23 - DNA, 22 bp, Artificial Sequence
  Ck12-2PS4 bioteg Autocatalytic template
  misc_feature (8)..(8): n is u
caatgacncctgcaatgactcc

SEQ ID NO: 24 - DNA, 21 bp, Artificial Sequence
  Cba12-2biot3 autocatalytic template
tcgtcagaatgctcgtcagaa

SEQ ID NO: 25 - DNA, 17 bp, Artificial Sequence
  ptBa12A5SP pseudotemplate
aaaaactcgtcagaatg

SEQ ID NO: 26 - DNA, 22 bp, Artificial Sequence
  Cbe12-2S4P autocatalytic template
cgatcctgaatgcgatcctgaa

SEQ ID NO: 27 - DNA, 16 bp, Artificial Sequence
  ApTBe12A3S3P pseudotemplate
aaacgatcctgaatga

SEQ ID NO: 28 - DNA, 22 bp, Artificial Sequence
  CBe12-2noPS3 autocatalytic template
cgatcctgaatgcgatcctgaa

SEQ ID NO: 29 - DNA, 22 bp, Artificial Sequence
  Ck12-2S4noUbioteg autocatalytic template
  misc_feature (8)..(8): n is u
caatgacncctgcaatgactcc

SEQ ID NO: 30 - DNA, 18 bp, Artificial Sequence
  ApTk12A5S3P pseudotemplate
  misc_feature (13)..(13): n is u
aaaaacaatgacncctga

SEQ ID NO: 31 - DNA, 21 bp, Artificial Sequence
  CBe12-2AULP autocatalytic template
cgatcctgaatgcgatcctga

SEQ ID NO: 32 - DNA, 48 bp, Artificial Sequence
  D21tof5TBe12S3P conversion template
cgatcctgaaagcgaagtttgactcatcaacatcagtctgataagcta

SEQ ID NO: 33 - DNA, 21 bp, Artificial Sequence
  CBe12-3noPS3 autocatalytic template
cgatcctgaatgcgatcctga

SEQ ID NO: 34 - DNA, 34 bp, Artificial Sequence
  D21tofBe12S0P conversion template
cgatcctgaatgtcaacatcagtctgataagcta

SEQ ID NO: 35 - DNA, 26 bp, Artificial Sequence
  CBe12-2SPCy355 autocatalytic template
cgatcctgaatgcgatccatcctgaa

SEQ ID NO: 36 - DNA, 22 bp, Artificial Sequence
  CBc12SPBMN35 autocatalytic template
cagtccagaatgcagtccagaa

SEQ ID NO: 37 - DNA, 17 bp, Artificial Sequence
  pTBc12T5SP pseudotemplate
tttttcagtccagaatg

SEQ ID NO: 38 - DNA, 48 bp, Artificial Sequence
  92atoF5TBe12PS0 conversion template
cgatcctgaaagcgaagtttgactcaagcattgcaaccgatcccaacc

SEQ ID NO: 39 - DNA, 48 bp, Artificial Sequence
  Let7atof5TBa12S0P conversion template
ctcgtcagaaagcgaagtttgactcaaactatacaacctactacctca

SEQ ID NO: 40 - DNA, 30 bp, Artificial Sequence
  CBa12-2AULP autocatalytic template
ctcgtcagaatgctcgtcagaaagcgaagc

SEQ ID NO: 41 - DNA, 16 bp, Artificial Sequence
  ApTBa12A3S3P pseudotemplate
aaactcgtcagaatga

SEQ ID NO: 42 - DNA, 28 bp, Artificial Sequence
  RPBe-Cy6 reporting probe
ttttgcattcaattttcgatcctgaatg

SEQ ID NO: 43 - DNA, 23 bp, Artificial Sequence
  CBe12-1S4 bioteg autocatalytic template
cgatcctgaatgcgatcctgaat

SEQ ID NO: 44 - DNA, 24 bp, Artificial Sequence
  Cba12S bioteg autocatalytic template
ctcgtcagaatgctcgtcagaatg

SEQ ID NO: 45 - DNA, 28 bp, Artificial Sequence
  RPBa-Hex reporting probe
ttttgaattctattttctcgtcagaatt

SEQ ID NO: 46 - DNA, 21 bp, Artificial Sequence
  Cba12-3noPS3 autocatalytic template
ctcgtcagaatgctcgtcaga

SEQ ID NO: 47 - DNA, 20 bp, Artificial Sequence
  RPBe-Cy5(2) reporting probe
ttcaggttttcgatcctgaa

SEQ ID NO: 48 - DNA, 21 bp, Artificial Sequence
  RPBe-Cy5(3) reporting probe
attcagaatgcgatcctgaat

SEQ ID NO: 49 - DNA, 18 bp, Artificial Sequence
  ptBa12A6biot pseudo-template
aaaaaactcgtcagaatg

SEQ ID NO: 50 - DNA, 45 bp, Artificial Sequence
  92atof1Be12-3+3 conversion template
atgcgatcctgacgtttgactcaagcattgcaaccgatcccaacc

SEQ ID NO: 51 - DNA, 45 bp, Artificial Sequence
  Let7atof1Ba12-3+3 conversion template
atgctcgtcagacgtttgactcaaactatacaacctactacctca

SEQ ID NO: 52 - DNA, 21 bp, Artificial Sequence
  CBc12-3noPS3 autocatalytic template
cagtccagaatgcagtccaga

SEQ ID NO: 53 - DNA, 20 bp, Artificial Sequence
  RPBc-FAM reporting probe
ttctggttttcagtccagaa

SEQ ID NO: 54 - DNA, 22 bp, Artificial Sequence
  alphatoalpha autocatalytic template
cagtccagaatgcagtccagaa

SEQ ID NO: 55 - DNA, 17 bp, Artificial Sequence
  pTalpha pseudo template
tttttcagtccagaatg

SEQ ID NO: 56 - DNA, 21 bp, Artificial Sequence
  rTalpha reporting template
attctgaatgcagtccagaat

SEQ ID NO: 57 - DNA, 44 bp, Artificial Sequence
  Let7atoalpha conversion template
tgcagtccagaagtttgactcaaactatacaacctactacctca

SEQ ID NO: 58 - DNA, 44 bp, Artificial Sequence
  Let7ctoalpha conversion template
tgcagtccagaagtttgactcaaaccatacaacctactacctca

SEQ ID NO: 59 - DNA, 39 bp, Artificial Sequence
  mir39toalpha conversion template
tgcagtccagaagtttgactcacaagctgatttacaccc

SEQ ID NO: 60 - DNA, 12 bp, Artificial Sequence
  bc trigger
cattctggactg

SEQ ID NO: 61 - DNA, 22 bp, Artificial Sequence
  aTc aT
cagtccagaatgcagtccagaa

SEQ ID NO: 62 - DNA, 17 bp, Artificial Sequence
  pTbc pT
tttttcagtccagaatg

SEQ ID NO: 63 - DNA, 21 bp, Artificial Sequence
  rTbc rT
attctgaatgcagtccagaat

SEQ ID NO: 64 - DNA, 36 bp, Artificial Sequence
  rTbc-biot rT
tttttttttgtgagaatgcagtccagaatgtctcac

SEQ ID NO: 65 - DNA, 51 bp, Artificial Sequence
  cTbc-Let7a-biot cT
tgcagtccagaagtttgactcaaactatacaacctactacctcattttttt

SEQ ID NO: 66 - DNA, 51 bp, Artificial Sequence; identical to SEQ ID NO: 65
  cTbc-92a-biot cT

SEQ ID NO: 67 - DNA, 52 bp, Artificial Sequence
  cTbc-203a-biot cT
tgcagtccagaagtttgactcaactagtggtcctaaacatttcacttttttt

SEQ ID NO: 68 - DNA, 44 bp, Artificial Sequence; identical to SEQ ID NO: 57
  cTbc-Let7a cT

SEQ ID NO: 69 - RNA, 22 nt, Artificial Sequence
  Let7a microRNA
ugagguaguagguuguauaguu

SEQ ID NO: 70 - RNA, 23 nt, Artificial Sequence
  mir92a microRNA
agguugggaucgguugcaaugcu

SEQ ID NO: 71 - RNA, 22 nt, Artificial Sequence
  mir203a-3p microRNA
gugaaauguuuaggaccacuag

SEQ ID NO: 72 - DNA, 22 bp, Artificial Sequence
  Let7a-D
tgaggtagtaggttgtatagtt

SEQ ID NO: 73 - DNA, 23 bp, Artificial Sequence
  mir92a-D microRNA
aggttgggatcggttgcaatgct

SEQ ID NO: 74 - DNA, 22 bp, Artificial Sequence
  mir203a-D microRNA
gtgaaatgtttaggaccactag

SEQ ID NO: 75 - DNA, 50 bp, Artificial
  16-5toBc T5 T7
tgcagtccagaagtttgactctttttcgccaatatttacgtgcttttttt

SEQ ID NO: 76 - DNA, 49 bp, Artificial
  10atoBc T5 T7
tgcagtccagaagtttgactctttttcacaaattcggatctacagggta

SEQ ID NO: 77 - DNA, 48 bp, Artificial
  203atoBc T5 T7
tgcagtccagaagtttgactctttttctagtggtcctaaacatttcac

SEQ ID NO: 78 - DNA, 54 bp, Artificial
  Lin4toBc T5 T7
tgcagtccagaagtttgactcttttttcacacttgaggtctcagggattttttt

SEQ ID NO: 79 - DNA, 54 bp, Artificial
  Let7atoBc T5 T7
tgcagtccagaagtttgacctttttaactatacaacctactacctcattttttt

SEQ ID NO: 80 - DNA, 55 bp, Artificial
  21toBc T5 T7
tgcagtccagaagtttgactcttttttcaacatcagtctgataagctattttttt

SEQ ID NO: 81 - DNA, 60 bp, Artificial
  21toBc T10 T7
tgcagtccagaagtttgactctttttttttttcaacatcagtctgataagctattttttt

SEQ ID NO: 82 - DNA, 65 bp, Artificial
  21toBc T15 T7
tgcagtccagaagtttgactcttttttttttttttttcaacatcagtctgataagctattttttt